(19) United States
(12) Patent Application Publication
(10) Pub. No.: US 2006/0292566 A1
(43) Pub. Date: Dec. 28, 2006

(54) Method for optimising gene expressing using synonymous codon optimisation

(75) Inventor: Ian Hector Frazer, St. Lucia, Queensland (AU)

Correspondence Address: HELLER EHRMAN WHITE & MCAULIFFE LLP, 1717 RHODE ISLAND AVE, NW, WASHINGTON, DC, 20036-3001, US

(73) Assignee: The University of Queensland, St. Lucia, Queensland (AU)

(21) Appl. No.: 10/534,130

(22) PCT Filed: Nov. 10, 2003
(86) PCT No.: PCT/AU03/01487

§ 371 Date: Dec. 30, 2005

Related U.S. Application Data
(60) Provisional application No. 60/425,163, filed on Nov. 8, 2002.

(51) Int. Cl. C12Q 1/68; A61K 48/00; C12N 15/74; C12N 1/21

(52) U.S. Cl. 435/6; 435/471; 435/252.3; 435/325; 514/44

(57) ABSTRACT

The present invention discloses a method for modulating the quality of a selected phenotype that is displayed by an organism or part thereof and that results from the expression of a polypeptide-encoding polynucleotide by replacing at least one codon of that polynucleotide with a synonymous codon that has a higher or lower preference of usage by the organism or part thereof to produce the selected phenotype than the codon it replaces. The present invention is also directed to the use of a codon-modified polynucleotide so constructed for modulating the quality of a selected phenotype displayed by an organism or part thereof.

### FIELD OF THE INVENTION

The present invention relates generally to gene expression. More particularly, the present invention relates to a method for modulating the quality of a selected phenotype that is displayed by an organism or part thereof and that results from the expression of a polynucleotide that encodes the polypeptide by replacing at least one codon of that polynucleotide with a synonymous codon that has a higher or lower preference of usage by the organism or part thereof to produce the selected phenotype than the codon it replaces. Even more particularly, the invention relates to the use of a protein-encoding polynucleotide whose codon composition has been modified for modulating the quality of a selected phenotype displayed by an organism or part thereof.

### BACKGROUND OF THE INVENTION

The expression of foreign heterologous genes in transformed cells is now commonplace. A large number of mammalian genes, including, for example, murine and human genes, have been successfully expressed in various host cells, including bacterial, yeast, insect, plant and mammalian host cells. Nevertheless, despite the burgeoning knowledge of expression systems and recombinant DNA technology, significant obstacles remain when one attempts to express a foreign or synthetic gene in a selected host cell. For example, translation of a synthetic gene, even when coupled with a strong promoter, often proceeds much more slowly than would be expected. The same is frequently true of exogenous genes that are foreign to the host cell. This lower than expected translation efficiency is often due to the protein coding regions of the gene having a codon usage pattern that does not resemble those of highly expressed genes in the host cell. It is known in this regard that codon utilisation is highly biased and varies considerably in different organisms and that biases in codon usage can alter peptide elongation rates. It is also known that codon usage patterns are related to the relative abundance of tRNA isoacceptors, and that genes encoding proteins of high versus low abundance show differences in their codon preferences.

The implications of codon preference phenomena on gene expression are manifest in that these phenomena can affect the translational efficiency of messenger RNA (mRNA). It is widely known in this regard that translation of “rare codons”, for which the corresponding iso-tRNA is in low abundance relative to other iso-tRNAs, may cause a ribosome to pause during translation which can lead to a failure to complete a nascent polypeptide chain and an uncoupling of transcription and translation. Thus, the expression of an exogenous gene may be impeded severely if a particular host cell of an organism or the organism itself has a low abundance of iso-tRNAs corresponding to one or more codons of the exogenous gene. Accordingly, a major aim of investigators in this field is to first ascertain the codon preference for particular cells in which an exogenous gene is to be expressed, and to subsequently alter the codon composition of that gene for optimised expression in those cells.

Codon-optimisation techniques are known for improving the translational kinetics of translationally inefficient protein coding regions. Traditionally, these techniques have been based on the replacement of codons that are rarely or infrequently used in the host cell with those that are host-preferred. Codon frequencies can be derived from literature sources for the highly expressed genes of many organisms (see, for example, Nakamura et al., 1996, Nucleic Acids Res 24: 214-215). These frequencies are generally expressed on an ‘organism-wide average basis’ as the percentage of occasions that a synonymous codon is used to encode a corresponding amino acid across a collection of protein-encoding genes of that organism, which are preferably highly expressed.

Typically, codons are classified as: (a) “common” codons (or “preferred” codons) if their frequency of usage is above about 4/3× the frequency of usage that would be expected in the absence of any bias in codon usage; (b) “rare” codons (or “non-preferred” codons) if their frequency of usage is below about ⅔× the frequency of usage that would be expected in the absence of any bias in codon usage; and (c) “intermediate” codons (or “less preferred” codons) if their frequency of usage is in-between the frequency of usage of “common” codons and of “rare” codons. Since an amino acid can be encoded by 2, 3, 4 or 6 codons, the frequency of usage of any selected codon, which would be expected in the absence of any bias in codon usage, will be dependent upon the number of synonymous codons which code for the same amino acid as the selected codon. Accordingly, for a particular amino acid, the frequency thresholds for classifying codons in the “common”, “intermediate” and “rare” categories will be dependent upon the number of synonymous codons for that amino acid. Consequently, for amino acids having 6 choices of synonymous codon, the frequency of codon usage that would be expected in the absence of any bias in codon usage is 16% and thus the “common”, “intermediate” and “rare” codons are defmed as those codons that have a frequency of usage above 20%, between 10 and 20% and below 10%, respectively. For amino acids having 4 choices of synonymous codon, the frequency of codon usage that would be expected in the absence of codon usage bias is 25% and thus the “common”, “intermediate” and “rare” codons are defined as those codons that have a frequency of usage above 33%, between 16 and 33% and below 16%, respectively. For isoleucine, which is the only amino acid having 3 choices of synonymous codon, the frequency of codon usage that would be expected in the absence of any bias in codon usage is 33% and thus the “common”, “intermediate” and “rare” codons for isoleucine are defined as those codons that have a frequency of usage above 45%, between 20 and 45% and below 20%, respectively. For amino acids having 2 choices of synonymous codon, the frequency of codon usage that would be expected in the absence of codon usage bias is 50% and thus the “common”, “intermediate” and “rare” codons are defined as those codons that have a frequency of usage above 60%, between 30 and 60% and below 30%, respectively. Thus, the categorisation of codons into the “common”, “intermediate” and “rare” classes (or “preferred”, “less preferred” or “non preferred”, respectively) has been based conventionally on a compilation of codon usage for an organism in general (e.g. ‘human-wide’) or for a class of organisms in general (e.g., ‘mammal-wide’). For example, reference may be made to Seed (see U.S. Pat. Nos. 5,786,464 and 5,795,737) who discloses preferred, less preferred and non-preferred codons for mammalian cells in general. However, the present inventor revealed in WO 99/02694 and in WO 00/42190 that there are substantial differences in the relative abundance of particular iso-tRNAs in different cells or tissues of a single multicellular organism (e.g., a mammal or a plant) and that this plays a pivotal role in protein translation from a coding sequence with a given codon usage or composition.

Thus, in contrast to the art-recognised presumption that different cells of a multicellular organism have the same bias in codon usage, it was revealed for the first time that one cell type of a multicellular organism uses codons in a manner distinct from another cell type of the same organism. In other words, it was revealed that different cells of an organism can exhibit different translational efficiencies for the same codon and that it was not possible to predict which codons would be preferred, less preferred or non preferred in a selected cell type. Accordingly, it was proposed that differences in codon translational efficiency between cell types could be exploited, together with codon composition of a gene, to regulate the production of a protein in, or to direct that production to, a chosen cell type.

Therefore, in order to optimise the expression of a protein-encoding polynucleotide in a particular cell type, it is necessary to first determine the translational efficiency for each codon in that cell type, rather than to rely on codon frequencies calculated on an organism-wide average basis, and then to codon modify the polynucleotide based on that determination.

All of the above methods relate to the codon optimisation of protein-encoding polynucleotides for modulating the expression of those polynucleotides in a chosen cell type or for differentially expressing those polynucleotides between selected cell types.

### BRIEF SUMMARY OF THE INVENTION

The present invention relates to a novel strategy for changing the quality of a selected phenotype that is displayed by an organism of interest and that results from the expression of a polynucleotide that encodes a polypeptide. In contrast to methods heretofore described, this strategy does not rely on codon usage data or on codon translational efficiency data that may be applicable to the organism of interest. Instead, it relies on ranking individual synonymous codons that code for an amino acid in the polypeptide according to their preference of usage by the organism of interest for producing the selected phenotype. In other words, the subject method is based on determining the “phenotypic preferences” of individual synonymous codons. Advantageously, phenotypic preferences can be determined by introducing into the organism of interest or into a related organism a synthetic construct that comprises a regulatory polynucleotide operably linked to a tandem repeat of a codon fused in frame with a reporter polynucleotide that encodes a protein, which produces, or which is predicted to produce, the selected phenotype or a phenotype of the same class as the selected phenotype. The quality of the phenotype produced by the expression of the synthetic construct in the organism of interest or in the related organism is then determined using a suitable assay. The selected phenotype may be a therapeutic or prophylactic phenotype including immunity, tolerance, pathogen resistance etc, or other beneficial trait including enhancement or prevention of a repair process, pest resistance, frost resistance, herbicide tolerance etc. In accordance with the present invention, a set of synonymous codons will often display a range of phenotypic preferences, which can be used as a basis for rationally selecting a codon in the original polynucleotide for replacement with a synonymous codon that has a different phenotypic preference.

Thus, in one aspect of the present invention, methods are provided for constructing a synthetic polynucleotide from which a polypeptide is producible to confer a selected phenotype upon an organism of interest or part thereof in a different quality than that conferred by a parent polynucleotide that encodes the same polypeptide. These methods generally comprise: (a) selecting a first codon of the parent polynucleotide for replacement with a synonymous codon, wherein the synonymous codon is selected on the basis that it exhibits a different phenotypic preference than the first codon in a comparison of phenotypic preferences in test organisms or parts thereof, wherein the test organisms are selected from the group consisting of organisms of the same species as the organism of interest and organisms that are related to the organism of interest; and (b) replacing the first codon with the synonymous codon to construct the synthetic polynucleotide

The phenotypic preferences of codons in the test organisms or parts are suitably compared by: (i) separately introducing into the test organisms or parts individual synthetic constructs, each of which comprises a regulatory polynucleotide operably linked to a tandem repeat of a codon fused in frame with a reporter polynucleotide that encodes a reporter protein, which produces, or which is predicted to produce, a corresponding phenotype selected from the group consisting of the selected phenotype and a phenotype of the same class as the selected phenotype; and (ii) comparing the quality of the phenotypes displayed by the test organisms or parts to determine the relative phenotypic preferences of the codons.

The synthetic constructs are typically introduced into the test organisms or parts using the same or similar mode of introduction. This is desirable when the corresponding phenotype or its quality is dependent on a particular mode or site of introduction of the synthetic constructs.

In some embodiments, the tandem repeat of each of the synthetic constructs comprises at least three copies of the corresponding codon.

In some embodiments, the synonymous codon is selected such that it has a higher phenotypic preference than the first codon. In accordance with the present invention, a higher phenotypic preference will correlate with a higher quality of the selected phenotype. Accordingly, a synonymous codon for these embodiments is preferably selectable when the quality of the phenotype conferred by the synthetic construct comprising a tandem repeat of the synonymous codon is suitably at least about 5% higher than the quality of the phenotype conferred by the synthetic construct comprising a tandem repeat of the first codon.

In other embodiments, the synonymous codon is selected such that it has a lower phenotypic preference than the first codon. According to the subject invention, a lower phenotypic preference will correlate with a lower quality of the selected phenotype. Thus, a synonymous codon for these embodiments is preferably selectable when the quality of the phenotype conferred by the synthetic construct comprising a tandem repeat of the synonymous codon is suitably at least about 5% lower than the quality of the phenotype conferred by the synthetic construct comprising a tandem repeat of the first codon.

Although the present invention extends to unicellular organisms, it is preferably applicable to multicellular organisms including plants and animals.

In another aspect, the invention provides methods for determining the phenotypic preference of a first codon in an organism of interest or part thereof. These methods generally comprise: (a) introducing a synthetic construct into a test organism or part thereof, wherein the test organism is selected from the group consisting of an organism of the same species as the organism of interest and an organism that is related to the organism of interest, the synthetic construct comprising a regulatory polynucleotide operably linked to a tandem repeat of the first codon fused in frame with a reporter polynucleotide that encodes a reporter protein, which produces, or which is predicted to produce, a selected phenotype or a phenotype of the same class as the selected phenotype; and (b) determining the quality of the corresponding phenotype displayed by the organism or part.

In some embodiments, the methods further comprise: comparing (i) the quality of the corresponding phenotype displayed by a test organism or part to which a synthetic construct comprising a tandem repeat of the first codon was provided; and (ii) the quality of the corresponding phenotype displayed by a test organism or part to which a synthetic construct comprising a tandem repeat of a second codon was provided, wherein the second codon encodes the same amino acid as the first codon, to thereby determine the phenotypic preference of the first codon relative to the phenotypic preference of the second codon in the test organism or part.

In some embodiments, the methods further comprise: (1) introducing the synthetic construct into a progenitor of a test organism or part; and (2) producing the test organism or part from the progenitor, wherein the test organism or part contains the synthetic construct.

In other embodiments, the methods further comprise: (1) introducing the synthetic construct into a progenitor of a test organism or part; and (2) growing the test organism or part from the progenitor; wherein the test organism or part comprises a cell containing the synthetic construct.

In still other embodiments, the methods further comprise: introducing the synthetic construct into a selected cell of the test organism or part.

In yet another aspect, the invention provides a synthetic polynucleotide constructed according to any one of the above methods.

In even yet another aspect, the invention encompasses an organism of interest or part thereof containing a synthetic polynucleotide constructed according to any one of the above methods.

In still another aspect, the invention contemplates an organism of interest or part thereof containing a synthetic construct that comprises a regulatory polynucleotide operably linked to a tandem repeat of a first codon fused in frame with a reporter polynucleotide that encodes a reporter protein, which produces, or which is predicted to produce, a selected phenotype or a phenotype of the same class as the selected phenotype.

In a further aspect, the invention embraces methods of modulating the quality of a selected phenotype that is displayed by an organism of interest or part thereof and that results from the expression of a parent polynucleotide that encodes the polypeptide. These methods generally comprise: introducing into the organism or part a synthetic polynucleotide that is distinguished from the parent polynucleotide by the replacement of a first codon in the parent polynucleotide with a synonymous codon that exhibits a different phenotypic preference than the first codon in the organism or part.

In yet a further aspect, the invention encompasses methods of enhancing the quality of a selected phenotype that is displayed by an organism of interest or part thereof and that results from the expression of a parent polynucleotide that encodes the polypeptide. These methods generally comprise: introducing into the organism or part a synthetic polynucleotide that is distinguished from the parent polynucleotide by the replacement of a first codon in the parent polynucleotide with a synonymous codon that exhibits a higher phenotypic preference than the first codon in the organism or part.

In even yet a further aspect, the invention extends to methods of reducing the quality of a selected phenotype that is displayed by an organism of interest or part thereof and that results from the expression of a parent polynucleotide that encodes the polypeptide. These methods generally comprise: introducing into the organism or part a synthetic polynucleotide that is distinguished from the parent polynucleotide by the replacement of a first codon in the parent polynucleotide with a synonymous codon that exhibits a lower phenotypic preference than the first codon in the organism or part.

### DETAILED DESCRIPTION OF TIRE INVENTION

### 1. DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “about” is used herein to refer to parameters (e.g., amounts, concentrations, phenotype-conferring efficiencies, time etc) that vary by as much as 30%, 20%, 15%, 10%, 5% or even 4%, 3%, 2%, 1% to a specified parameter.

As used herein, the term “cis-acting sequence” or “cis-regulatory region” or similar term shall be taken to mean any sequence of nucleotides which is derived from an expressible genetic sequence wherein the expression of the genetic sequence is regulated, at least in part, by said sequence of nucleotides. Those skilled in the art will be aware that a cis-regulatory region may be capable of activating, silencing, enhancing, repressing or otherwise altering the level of expression and/or cell-type-specificity and/or developmental specificity of any structural gene sequence.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein a “conferred phenotype” refers to a temporary or permanent change in the state of an organism of interest or class of organisms of interest, or of a part or tissue or cell or cell type or class of cell of an organism of interest, which occurs after the introduction of a polynucleotide to that organism, or to that class of organisms, or to the part or tissue or cell or cell type or class of cell, or to a precursor of that organism or part or tissue or cell or cell type or class of cell, and which would not have occurred in the absence of that introduction. Typically, such a temporary or permanent change occurs as a result of the transcription and/or translation of genetic information contained within that polynucleotide in the cell, or in at least one cell or cell type or class of cell within the organism of interest or within the class of class of organisms of interest, and can be used to distinguish the organism of interest, or class of organisms of interest, or part or tissue or cell or cell type or class of cell thereof, or genetic progeny of these, to which the polynucleotide has been provided from a similar organism of interest, or class of organisms of interest, or part or tissue or cell or cell type or class of cell thereof, or genetic progeny of these, to which the polynucleotide has not been provided.

By “expression vector” is meant any autonomous genetic element capable of directing the synthesis of a protein encoded by the vector. Such expression vectors are known by practitioners in the art.

The term “gene” as used herein refers to any and all discrete coding regions of a host genome, or regions that code for a functional RNA only (e.g., tRNA, rRNA, regulatory RNAs such as ribozymes, post translational gene silencing-associated RNAs etc) as well as associated non-coding regions and optionally regulatory regions. In certain embodiments, the term “gene” gene includes within its scope the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The gene sequences may be cDNA or genomic DNA or a fragment thereof. The gene may be introduced into an appropriate vector for extrachromosomal maintenance or for integration into the host.

By “modulating”, “modulate” and the like is meant increasing or decreasing, either directly or indirectly, the quality of a selected phenotype. 20

By “natural gene” is meant a gene that naturally encodes the protein. However, it is possible that the parent polynucleotide encodes a protein that is not naturally-occurring but has been engineered using recombinant techniques.

The term “5′ non-coding region” is used herein in its broadest context to include all nucleotide sequences which are derived from the upstream region of an expressible gene, other than those sequences which encode amino acid residues which comprise the polypeptide product of said gene, wherein 5′ non-coding region confers or activates or otherwise facilitates, at least in part, expression of the gene.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphorarnidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

The term “operably connected” or “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e. the genes from which it is derived.

The terms “precursor” and “progenitor” as used herein refer to a cell or part of organism that can gives rise to an organism of interest in which phenotypic expression is desired or in which phenotypic preference of a codon is to be determined.

The term “phenotype” means any one or more detectable physical or functional characteristics, properties, attributes or traits of an organism, tissue, or cell, or class of organisms, tissues or cells, which generally result from the interaction between the genetic makeup (i.e., genotype) of the organism, tissue, or cell, or the class of organisms, tissues or cells and the environment. In certain embodiments, the term “phenotype” excludes resistance to a selective agent or screening an enzymic or light-emitting activity, conferred directly by a reporter protein.

By “phenotypic preference” is meant the preference with which an organism uses a codon to produce a selected phenotype. This preference can be evidenced, for example, by the quality of a selected phenotype that is producible by a polynucleotide that comprises the codon in an open reading frame which codes for a polypeptide that produces the selected phenotype. In certain embodiment, the preference of usage is independent of the route by which the polynucleotide is introduced into the organism. However, in other embodiments, the preference of usage is dependent on the route of introduction of the polynucleotide into the organism.

The term “polynucleotide” or “nucleic acid” as used herein designates mRNA, RNA, cRNA, cDNA or DNA. The term typically refers to oligonucleotides greater than 30 nucleotides in length.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

By “producing”, and like terms such as “production” and “producible”, in the context or protein production, is meant production of a protein to a level sufficient to achieve a particular function or phenotype associated with the protein. By contrast, the terms “not producible” and “not substantially producible” as used interchangeably herein refer to (a) no production of a protein, (b) production of a protein to a level that is not sufficient to effect a particular function or phenotype associated with the protein, (c) production of a protein, which cannot be detected by a monoclonal antibody specific for the protein, or (d) production of a protein, which is less that 1% of the level produced in a wild-type cell that normally produces the protein.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e. upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The term “quality” is used herein in its broadest sense and includes a measure, strength, intensity, degree or grade of a phenotype, e.g., a superior or inferior immune response, increased or decreased disease resistance, higher or lower sucrose accumulation, better or worse salt tolerance etc.

By “recombinant polypeptide” is meant a polypeptide made using recombinant techniques, i.e., through the expression of a recombinant or synthetic polynucleotide.

The term “synthetic polynucleotide” as used herein refers to a polynucleotide formed in vitro by the manipulation of a polynucleotide into a form not normally found in nature. For example, the synthetic polynucleotide can be in the form of an expression vector. Generally, such expression vectors include transcriptional and translational regulatory polynucleotide operably linked to the polynucleotide.

The term “synonymous codon” as used herein refers to a codon having a different nucleotide sequence than another codon but encoding the same amino acid as that other codon.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

2. Phenotypic Preference of Codons

The present invention provides a novel strategy for enhancing or reducing the quality of a selected phenotype that is displayed, or proposed to be displayed, by an organism of interest. The strategy involves codon modification of a polynucleotide that encodes a phenotype-associated polypeptide that either by itself or in association with other molecules, in the organism of interest imparts or confers the selected phenotype upon the organism. Unlike previous methods, however, the strategy does not rely on data that provide a ranking of synonymous codons according to their preference of usage in an organism or class of organisms. Nor does it rely on data that provide a ranking of synonymous codons according to their translational efficiencies in one or more cells of the organism or class of organisms. Instead, it relies on ranking individual synonymous codons that code for an amino acid in the phenotype-associated polypeptide according to their preference of usage by the organism or class of organisms, or by a part thereof, for producing the selected phenotype. As used herein, the preference of usage of an individual codon by an organism or class of organisms or by a part thereof is referred to as a “phenotypic preference”. Such preference may be determined by qualitatively or quantitatively determining the influence of the individual codon on a achieving the selected phenotype and it does not require, therefore, the calculation of codon frequencies or translational efficiencies as heretofore described.

Advantageously, phenotypic preferences for synonymous codons can be determined by introducing into the organism of interest or into a related organism, or into a part of such organisms a synthetic construct that comprises a regulatory polynucleotide operably linked to a tandem repeat of a codon fused in frame with a reporter polynucleotide that encodes a reporter protein, which produces, or which is predicted to produce, the selected phenotype or a phenotype of the same class as the selected phenotype. In one embodiment, the reporter protein is a phenotype-associated polypeptide (e.g., a melanoma specific antigen such as BAGE or GAGE-1) that will be the subject of producing the selected phenotype (e.g., immunity to melanoma). In another embodiment, the phenotype-associated polypeptide (e.g., green fluorescent protein or a gastrointestinal associated antigen such as 17-1A) is incapable of producing the selected phenotype (e.g., immunity to melanoma) but produces the same class of phenotype (e.g., an immune response) as the selected phenotype. In illustrative examples, the phenotype-associated polypeptide is selected from antigens including antigens from pathogenic organisms or cancers (e.g., wherein the phenotype is immunity to disease) and self antigens or transplantation antigens (e.g., wherein the phenotype is antigen-specific anergy or tolerance), growth factors (e.g., wherein the phenotype is selected from size of the organism or part, wound healing, cell proliferation, cell differentiation, cell migration, immune cell function), hormones (e.g., wherein the phenotype is increased lactation, e.g., using oxytocin, or amelioration of a diabetic state, e.g., using insulin) and toxins (e.g., wherein the phenotype is tumour regression or cell death).

The design of the synthetic construct is preferably based on the synthetic construct design of Frazer et al. in International Publication WO 00/42215, which is predicated on the determination that different but synonymous stretches of identical codons fused respectively in frame with a reporter polynucleotide can give rise to different levels of reporter protein produced within a given cell type. The level of reporter protein produced in a selected cell type is sensitive to the intracellular abundance of the iso-tRNA species corresponding to the identical codons of the construct and, therefore, provide a direct correlation of a cell's preference for translating a given codon. However, in contrast to WO 00/42215, which is primarily concerned with determining translational efficiencies of synonymous codons in selected cell types, the present invention is concerned with using synthetic constructs to determine the influence of their synonymous stretches of identical codons on the phenotype or class of phenotype displayed by the organism or part in response to the phenotype-associated protein produced by those synthetic constructs. This means, for example, that if the quality of the phenotype displayed by a selected organism or part thereof to which a synthetic construct having a tandem series of identical first codons is provided is lower than the quality of the phenotype displayed by the selected organism or part to which a synthetic construct having a tandem series of identical second codons is provided (i.e., wherein the first codons are different than, but synonymous with, the second codons), then it can be deduced that the selected organism or part has a higher preference for the second codon than the first codon with respect to the quality of the phenotype produced. Put another way, the second codon has a higher phenotypic preference than the first codon in the selected organism or part.

Suitably, the tandem repeat comprises at least three identical codons, preferably between four and seven identical codons and more preferably five or six identical codons.

The tandem repeat can be fused at a location adjacent to, or within, the reporter polynucleotide. This location is preferably selected such that the tandem repeat interferes with translation of at least a portion of the phenotype-associated protein. Preferably, the tandem repeat is located immediately upstream (translationally) from the reporter polynucleotide.

It is of course possible that a tandem repeat of identical amino acid residues (e.g., an oligo-proline repeat) can render the phenotype-associated protein unstable. Typically, protein instability is detected when the phenotype conferred by expression of the reporter polynucleotide is not detectable with any choice of isoaccepting codon specific for the amino acid corresponding to the tandem repeat. However, such protein instability can be alleviated by use of at least one spacer codon within the tandem repeat of identical codons, wherein the spacer codon encodes a neutral amino acid.

The spacer codon(s) can be placed adjacent to, or interposed between, some or all of the identical codons corresponding to the tandem repeat. For example, a suitable interposition for a penta-repeat of identical codons can be selected from the group consisting of: (a) I-S-I-S-I-S-I-S-I-S; (b) S-I-S-I-S-I-S-I-S-I; (c) I-S-I-S-I-I-S-I; (d) I-S-I-I-S-I-S-I; (e) I-S-I-S-I-I-I; (f) I-I-S-I-S-I-I; (g) I-I-I-S-I-S-I; (h) I-S-I-I-S-I-I; (i) I-I-S-I-I-S-I-I; (j) I-S-I-I-I-S-I; (k) I-S-I-I-I-I; (l) I-I-S-I-I-I; I-I-I-S-I-I; and (n) I-I-I-I-S-I, wherein I corresponds to an identical codon of a tandem repeat and S corresponds to a spacer codon.

Preferably, a spacer codon is efficiently translated in a cell type of the organism or the organism itself relative to other synonymous codons. This is important so that translation of the spacer codon is not rate limiting. The neutral amino acid includes, but is not restricted to, alanine and glycine.

The reporter polynucleotide can encode any suitable protein that confers upon the organism or part, either by itself or in association with other molecules, a phenotype or class of phenotype. In specific embodiments, the selected phenotype or class of phenotype corresponds to a beneficial or improved or superior state or condition of the organism or part thereof relative to a reference state or condition. In illustrative examples, the reference state or condition corresponds to a pathophysiological state. Phenotypes contemplated by the present invention include any desirable beneficial trait including, but not restricted to: immunity (e.g., immunity to pathogenic infection or cancer); antigen tolerance (e.g., antigen-specific T lymphocyte anergy, tolerance to allergens, transplantation antigens and self antigens); angiogenesis (e.g., blood vessel formation in the heart and vasculature and in tumour growths); anti-angiogenesis (e.g., treatment of ischaemic heart disease and tumours); amelioration of clinical symptoms (e.g., fever; inflammation; encephalitis; weight loss; anaemia; sensory symptoms such as paraesthesia or hypaesthesia; ataxia; neuralgia; paralysis; vertigo; urinary or bowel movement abnormalities; and cognitive dysfunction such memory loss, impaired attention, problem-solving difficulties, slowed information processing, and difficulty in shifting between cognitive tasks); reduced or increased cell death (e.g., apoptosis); reduced or increased cell differentiation; reduced or increased cell proliferation; tumour or cancer regression; growth and repair of tissue or organ; decreased fibrosis; inhibition or reversal of cell senescence; increased or reduced cell migration; differential expression of protein between different cells or tissues of an organism or part thereof; trauma recovery; recovery from burns; antibiotic resistance or sensitivity (e.g., resistance or sensitivity to aminoglycosidic antibiotics such as geneticin and paromomycin); herbicide tolerance or sensitivity (e.g. tolerance or sensitivity to glyphosate or glufosinate); starch biosynthesis or modification (e.g. using a starch branching enzyme, starch synthases, ADP-glucose pyrophosphorylase); fatty acid biosynthesis (e.g. using a desaturase or hydroxylase); disease resistance or tolerance (e.g., resistance to animal diseases such as cardiovascular disease, autoimmunity, Alzheimer's disease, Parkinson's disease, diabetes, AIDS etc or resistance to plant diseases such as rust, dwarfism, rot, smut, mould, scab and mildew); pest resistance or tolerance including insect resistance or tolerance (e.g., resistance to borers and worms); viral resistance or tolerance (e.g. resistance to animal viruses such as herpesviruses, hepadnaviruses, adenoviruses, flaviviruses, lentiviruses, poxviruses etc or resistance to plant viruses such as badnaviruses, caulimoviruses, potyviruses, luteoviruses, rhabdoviruses etc); fungal resistance or tolerance (e.g., resistance to arbuscular mycorrhizal fungi, endophytic fungi etc); a metabolic trait including sucrose metabolism (e.g., sucrose isomerisation); frost resistance or tolerance; stress tolerance (e.g., salt tolerance, drought tolerance); and improved food content or increased yields. Persons of skill in the art will recognise that the above exemplary classes of phenotype may be subdivided into phenotypic subclasses and that such subclasses would also fall within the scope of phenotypic classes contemplated by the present invention. For example, subclasses of immunity include innate immunity (which can be further subdivided inter alia into complement system, monocytes, macrophages, neutrophils and natural killer cells), cellular immunity (which can be further subdivided inter alia into cytolytic T lymphocytes, dendritic cells and T helper lymphocytes) and humoral immunity (which can be further subdivided inter alia into antibody subclasses IgA, IgD, IgE, IgG and IgM).

The instant method is applicable to prokaryotic as well as eukaryotic hosts and includes unicellular organisms and especially to multicellular organisms, such as but not limited to yeast, plants and animals including vertebrate animals such as mammals, reptiles, fish, birds etc as well as invertebrate animals such as metazoa, sponges, worms, molluscs, nematodes, crustaceans, echinoderms etc. In certain embodiments of the present invention, the organism of interest is selected from plants and mammals.

illustrative examples of eukaryotic organisms include, but are not limited to, fungi such as yeast and filamentous fungi, including species of Aspergillus, Trichiodenna, and Neurospora; animal hosts including vertebrate animals illustrative examples of which include fish (e.g., salmon, trout, tulapia, tuna, carp, flounder, halobut, swordfish, cod and zebrafish), birds (e.g., chickens, ducks, quail, pheasants and turkeys, and other jungle foul or game birds) and mammals (e.g., dogs, cats, horses, cows, buffalo, deer, sheep, rabbits, rodents such as mice, rats, hamsters and guinea pigs, goats, pigs, primates, marine mammals including dolphins and whales, as well as cell lines, such as human or other mammalian cell lines of any tissue or stem cell type (e.g., COS, NIH 3T3 CHO, BHK, 293, or HeLa cells), and stem cells, including pluripotent and non-pluripotent and embryonic stem cells, and non-human zygotes), as well as invertebrate animals illustrative examples of which include nematodes (representative generae of which include those that infect animals such as but not limited to Ancylostoina, Ascaridia, Ascaris, Bunostomum, Caenorhabditis, Capillaria, Chabertia, Cooperia, Dictyocaulus, Haernonchus, Heterakis, Nematodirus, Oesophagostomunt, Ostertagia, Oxyuris, Parascaris, Strongylus, Toxascaris, Trichuris, Trichostrongylus, Tflichonema, Toxocara, Uncinaria, and those that infect plants such as but not limited to Bursaphalenchus, Criconerriella, Diiylenchus, Ditylenchus, Globodera, Helicotylenchus, Heterodera, Longidorus, Melodoigyne, Nacobbus, Paratylenchus, Pratylenchus, Radopholus, Rotelynchus, Tylenchus, and Xphinerna) and other worms, drosophila, and other insects (such as from the families Apidae, Curculionidae, Scarabaeidae, Tephritidae, Tortricidae, amongst others, representative orders of which include Coleoptera, Diptera, Lepidoptera, and Homoptera.

In certain embodiments, the organism of interest is a plant which is suitably selected from monocotyledons, dicotyledons and gymnosperms. The plant may be an ornamental plant or crop plant. Illustrative examples of ornamental plants include, but are not limited to, Malus spp, Crataegus spp, Rosa spp., Betula spp, Sorbus spp, Olea spp, Nerium spp, Salix spp, Populus spp. Illustrative examples of crop plants include plant species which are cultivated in order to produce a harvestable product such as, but not limited to, Abelmoschus esculentus (okra), Acacia spp., Agave fourcroydes (henequen), Agave sisalana (sisal), Albizia spp., Allium fistulosum (bunching onion), Allium sativum (garlic), Allium spp. (onions), Alpinia galanga (greater galanga), Amaranthus caudatus, Ainarantthus spp., Anacardiumn spp. (cashew), Ananas comosus (pineapple), Anethunm graveolens (dill), Annonia cherimola (cherimoya), Apios americana (American potatobean), Arachis hypogaea (peanut), Arctium spp. (burdock), Artemisia spp. (wormwood), Aspalathlus linearis (redbush tea), Athertonia diversifolia, Atriplex nummularia (old man saltbush), Averrhoa carambola (starfiuit), Azadirachta indica (neem), Backhousia spp., Bambusa spp. (bamboo), Beta vulgaris (sugar beet), Boehmeria nivea (ramie), bok choy, Boronia megastignma (sweet boronia), Brassica carinata (Abyssinian mustard), Brassica juncea (Indian mustard), Brassica napus (rapeseed), Brassica oleracea (cabbage, broccoli), Brassica oleracea var Albogabra (gai lum), Brassica parachliniensis (choi sum), Brassica pekensis (Wong bok or Chinese cabbage), Brassica spp., Burcella obovata, Cajaizus cajan (pigeon pea), Camellia sinentsis (tea), Cannabis sativa (non-drug hemp), Capsicum spp., Carica spp. (papaya), Carthamtus tinctorius (safflower), Carum carvi (caraway), Cassinzia spp., Castanospernum australe (blackbean), Casuarina cunningizainiana (beefwood), Ceratonia siliqua (carob), Chamaemelum nobile (chamomile), Chamelaucium spp. (Geraldton wax), Cheniopodium quinoa (quinoa), Chrysanthemumn (Tanacetum), cinerariifolium (pyrethrum), Cicer arietinum (chickpea), Cichorium intybus (chicory), Clematis spp., Clianthus formosus (Sturt's desert pea), Cocos nucifera (coconut), Coffea spp. (coffee), Colocasia esculenta (taro), Coriandrum sativum (coriander), Craimbe abyssinica (crambe), Crocus sativus (saffron), Cucurbita foetidissima (buffalo gourd), Cucurbita spp. (gourd), Cchlorobenzoic yamopsis tetragonoloba (guar), Cyinbopogon spp. (lemongrass), Cytisus proliferus (tagasaste), Daucus carota (carrot), Desmanthus spp., Dioscorea esculenta (Asiatic yam), Dioscorea spp. (yams), Diospyros spp. (persimmon), Doronicuin sp., Echinacea spp., Eleocharis dulcis (water chestnut), Eleusine coracana (finger millet), Emanthus arundinaceus, Eragrostis tef (tef), Erianthus arundinaceus, Eriobotiyajaponica (loquat), Eucalyptus spp., Eucalyptus spp. (gil mallee), Euclea spp., Eugenia malaccensis (umba), Euphorbia spp., Euphoria longana (longan), Eutrema wasabi (wasabi), Fagopyrum esculentum (buckwheat), Festuca arundinacea (tall fescue), Ficus spp. (fig), Flacourtia inermis, Flindersia grayliana (Queensland maple), Foeniculum olearia, Foeniculum vulgare (fennel), Garcinia inangostana (mangosteen), Glycine latifolia, Glycine max (soybean), Glycine max (vegetable soybean), Glycyrrhiza glabra (licorice), Gossypium: spp. (cottons), Grevillea spp., Grindelia spp., Guizotia abyssinica (niger), Harpagophyllum sp., Helianthus aiznuus (high oleic sunflowers), Helianthus annuus (monosun sunflowers), Helianthus tuberosus (Jerusalem artichoke), Hibiscus cannabinus (kenaf), Hordeuin bulbosuin, Hordeum spp. (waxy barley), Hordeum vulgare (barley), Hordeum vulgare subsp. spontaneum, Humulus lupulus (hops), Hydrastis canadensis (golden seal), Hymienachne spp., Hyssopus officiialis (hyssop), Indigofera spp., Inga edulis (ice cream bean), Inocarpus tugiter, Ipoinoea batatas (sweet potato), Ipomoea sp. (kang kong), Lablab purpureus (white lablab), Lactuca spp. (lettuce), Lathyrus spp. (vetch), Lavandula spp. (avender), Lens spp. (lentil), Lesquerella spp. (bladderpod), Leucaena spp., Lilium spp., Lininanthes spp. (meadowfoam), Linumi usitatissimum (flax), Linum usitatissimum (linseed), Linum usitatissimum (Linola.TM.), Litchi chinensis (lychee), Lotus corniculatus (birdsfoot trefoil), Lotus pedunculatus, Lotus sp., Luffa spp., Lunaria annua (honesty), Lupinus mutabilis (pearl lupin), Lupinus spp. (lupin), Macadamia spp., Mangifera indica (mango), Manihot esculenta (cassava), Medicago spp. (luceme), Medicago spp., Melaleuca spp. (tea tree), Melaleuca uncinata (broombush), Menthla tasmannia, Mentha spicata (spearmint), Mentha X piperita (peppermint), Momordica charantia (bitter melon), Musa spp. (banana), Myrciaria cauliflora (jaboticaba), Myrothiainnus flabellifolia, Nephelium lappaceum (rambutan), Nerine spp., Ocimum basilicum (basil), Oenanthe javanica (water dropwort), Oenothera biennis (evening primrose), Olea europaea (olive), Olearia sp., Origanum spp. (marjoram, oregano), Oryza spp. (rice), Oxalis tuberosa (oca), Ozothamnus spp. (rice flower), Pachyrrhlizus ahipa (yam bean), Panax spp. (ginseng), Panicum miliaceum (common millet), Papaver spp. (poppy), Partheniium argentatum (guayule), Passiflora sp., Paulowinia toinemtosa (princess tree), Pelargoniuzn graveolens (rose geranium), Pelargonium sp., Pennisetunm americanum (bulrush or pearl millet), Persoonia spp., Petroselinunm crispum (parsley), Phacelia tanacetifolia (tansy), Phalaris canariensis (canary grass), Phalapis sp., Plzaseolus coccineus (scarlet runner bean), Phaseolus lunatus (lima bean), Phaseolus spp., Phaseolus vulgaris (culinary bean), Phaseolus vulgaris (navy bean), Phaseolus vulgaris (red kidney bean), Pisum sativum (field pea), Plantago ovata (psyllium), Polygonuin minus, Polygonumn odoratuin, Prunus mume (Japanese apricot), Psidium guajava (guava), Psophocarpus tetragonolobus (winged bean), Pyrus spp. (nashi), Raphanus satulus (long white radish or Daikon), Rhzagodia spp. (saltbush), Ribes nigrum (black currant), Ricinus communis (castor bean), Rosmarinus officinalis (rosemary), Runigia klossii (rungia), Saccharuin officinarum (sugar cane), Salvia officinalis (sage), Salvia sclarea (clary sage), Salvia sp., Sandersonia sp., Santalum acuminatunm (sweet quandong), Santalum spp. (sandalwood), Sclerocarya caffra (marula), Scutellaria galericulata (scullcap), Secale cereale (rye), Sesamum inidicum (sesame), Setaria italica (foxtail millet), Simmondsia spp. (jojoba), Solanum spp., Sorghum almum (sorghum), Stachys betonica (wood betony), Stenanthemum scortecheni, Strychnos cocculoides (monkey orange), Stylosanthes spp. (stylo), Szgiun spp., Tasmannia lanceolata (mountain pepper), Tenninalia karnbachii, Theobroina cacao (cocoa), Thlymus vulgaris (thyme), Toona australis (red cedar), Tnifoliiun: spp. (clovers), Trifolium alexandrinum (berseem clover), Trifolium resupinatuin (persian clover), Triticuin spp., Triticum tauschii, Tylosema esculentum (morama bean), Valeriana sp. (valerian), Vernonia spp., Vetiver zizanioides (vetiver grass), Vicia benghalensis (purple vetch), Vicia faba (faba bean), Vicia narbonensis (narbon bean), Vicia sativa, Vicia spp., Vigna aconitifolia (mothbean), Vigna angularis (adzuki bean), Vigiia mungo (black gram), Vigna radiata (mung bean), Vigna spp., Vigna unguiculata (cowpea), Vitis spp. (grapes), Voandzeia subterranea (bambarra groundnut), Triticosecale (triticale), Zea mays (bicolour sweetcorn), Zea mays (maize), Zea mays (sweet corn), Zea mays subsp. mexicana (teosinte), Zieria spp., Zingiber officinale (ginger), Zizania spp. (wild rice), Ziziphus jujuba (common jujube). Desirable crops for the practice of the present invention include Nicotiana tabacunm (tobacco) and horticultural crops such as, for example, Ananas comosus (pineapple), Saccharunm spp (sugar cane), Musa spp (banana), Lycopersicon esculentum (tomato) and Solanum tuberosum (potato).

The synthetic constructs of the invention may be introduced directly into an organism of interest or into one or more of its parts, e.g., cell or tissue types (e.g., a muscle, skin, brain, lung, kidney, pancreas, a reproductive organ such as testes, ovaries and breast, eye, liver, heart, vascular cell, root, leaf, flower, stalk or meristem) or into an organ of the organism. Alternatively, the synthetic construct is introduced into a progenitor of the organism and the progenitor is then grown or cultured for a time and under conditions sufficient to produce the organism of interest, whereby the synthetic construct is contained in one or more cell types of that organism. Suitable progenitor cells include, but are not limited to, stem cells such as embryonic stem cell, pluripotential immune cells, meristematic cells and embryonic callus. In certain embodiments, the synthetic construct is introduced into the organism of interest using a particular route of administration (e.g, for mammals, by the oral, parenteral (e.g., intravenous, intramuscular, intraperitoneal, intaventricular, intrarticular), mucosal (e.g., intranasal, intrapulmonary, oral, buccal, sublingual, rectal, intravaginal), dermal (topical, subcutaneous, transdermal); for plants, administration to flowers, meristem, root, leaves or stalk). Practitioners in the art will recognise that the route of administration will differ depending on the choice of organism of interest and the sought-after phenotype. Desirably, the synthetic constructs are introduced into the same or corresponding site. In other embodiments, the synthetic construct is introduced into a cell of the organism of interest (e.g., autologous cells), or into a cell that is compatible with the organism of interest (e.g., syngeneic or allogeneic cells) and the genetically-modified cell so produced is introduced into the organism of interest at a selected site or into a part of that organism.

The synthetic constructs of the invention may be introduced into an organism of interest or part thereof using any suitable method, and the kind of method employed will differ depending on the intended cell type, part and/or organism of interest. For example, four general classes of methods for delivering nucleic acid molecules into cells have been described: (1) chemical methods such as calcium phosphate precipitation, polyethylene glycol (PEG)-mediate precipitation and lipofection; (2) physical methods such as microinjection, electroporation, acceleration methods and vacuum infiltration; (3) vector based methods such as bacterial and viral vector-mediated transformation; and (4) receptor-mediated. Transformation techniques that fall within these and other classes are well known to workers in the art, and new techniques are continually becoming known. The particular choice of a transformation technology will be determined by its efficiency to transform certain host species as well as the experience and preference of the person practising the invention with a particular methodology of choice. It will be apparent to the skilled person that the particular choice of a transformation system to introduce a synthetic construct of the invention into cells is not essential to or a limitation of the invention, provided it achieves an acceptable level of nucleic acid transfer. Thus, the synthetic constructs are introduced into tissues or host cells by any number of routes, including viral infection, phage infection, microinjection, electroporation, or fusion of vesicles, lipofection, infection by Agrobacteiium tumefaciens or A. rhlizogenes, or protoplast fusion. Jet injection may also be used for intramuscular administration (as described for example by Furth et al., 1992, Anal Biochem 205:365-368). The synthetic constructs may be coated onto microprojectiles, and delivered into a host cell or into tissue by a particle bombardment device, or “gene gun” (see, for example, Tang et al., 1992, Nature 356:152-154). Alternatively, the synthetic constructs can be fed directly to, or injected into, the host organism or it may be introduced into the cell (i.e., intracellularly) or introduced extracellularly into a cavity, interstitial space, into the circulation of an organism, introduced orally, etc. Methods for oral introduction include direct mixing of the synthetic constructs with food of the organism. In certain embodiments, a hydrodynamic nucleic acid administration protocol is employed (e.g., see Chang et al., 2001, J. Virol. 75:3469-3473; Liu et al., 1999, Gene Ther. 6:1258-1266; Wolff et al., 1990, Science 247:1465-1468; Zhang et al., 1999, Hum. Gene Ther. 10:1735-1737; and Zhang et al., 1999, Gene Ther. 7:1344-1349). Other methods of nucleic acid delivery include, but are not limited to, liposome-mediated transfer, naked DNA delivery (direct injection) and receptor-mediated transfer (Oigand-DNA complex).

In accordance with the present invention, the individual synthetic constructs are separately introduced into test organisms which are preferably selected from organisms of the same species as the organism of interest or organisms that are related to the organism of interest, or into test parts of such organisms. Related organisms are generally species within the same phylum, preferably species within the same subphylum, more preferably species within superclass, even more preferably species within the same class, even more preferably species within the same order and still even more preferably species within the same genus. For example, if the organism of interest is human, a related species is suitably selected from mouse, cow, dog or cat, which belong to the same class as human, or a chimpanzee, which belongs to the same order as human. Alternatively, if the organism of interest is banana, the related organism may be selected from taro, ginger, onions, garlic, pineapple, bromeliaeds, palms, orchids, lilies, irises and the like, which are all non-graminaceous monocotyledonous plants and which constitute horticultural or botanical relatives.

After introduction of the synthetic constructs into the test organisms or parts, the qualities of their phenotypes are determined by a suitable assay and then compared to determine the relative phenotypic preferences of the synonymous codons. The quality is suitably a measure of the strength, intensity or grade of the phenotype, or the relative strength, intensity or grade of two or more desired phenotypic traits.

Assays for various phenotypes conferred by the production of a chosen reporter protein are known by those of skill in the art. For example, immunity may be assayed by any suitable methods that detects an increase in an animal's capacity to respond to foreign or disease-specific antigens (e.g., cancer antigens) i.e., those cells primed to attack such antigens are increased in number, activity, and ability to detect and destroy the those antigens. Strength of immune response is measured by standard tests including: direct measurement of peripheral blood lymphocytes by means known to the art; natural killer cell cytotoxicity assays (see, e.g., Provinciali et al (1992, J. Inmunol Meth. 155: 19-24), cell proliferation assays (see, e.g., Vollenweider and Groseurth (1992, J. Immunol. Meth. 149: 133-135), immunoassays of immune cells and subsets (see, e.g., Loeffler et al. (1992, Cytom. 13: 169-174); Rivoltini et al. (1992, Can. Immunol. Immunother. 34: 241-251); or skin tests for cell-mediated immunity (see, e.g., Chang et al (1993, Cancer Res. 53: 1043-1050). Enhanced immune response is also indicated by physical manifestations such as fever and inflammation, as well as healing of systemic and local infections, and reduction of symptoms in disease, i.e., decrease in tumour size, alleviation of symptoms of a disease or condition including, but not restricted to, leprosy, tuberculosis, malaria, naphthous ulcers, herpetic and papillomatous warts, gingivitis, artherosclerosis, the concomitants of AIDS such as Kaposi's sarcoma, bronchial infections, and the like. Such physical manifestations may also be used to detect, or define the quality of, the phenotype or class of phenotype displayed by an organism. Alternatively, herbicide tolerance may be assayed by treating test organisms (e.g., plants such as cotton plants), which express a herbicide tolerance gene (e.g., glyphosate tolerance protein gene such as a glyphosate resistant EPSP synthase), with a herbicide (e.g., glyphosate) and determining the efficacy of herbicide tolerance displayed by the plants. For example, when determining the efficacy of synthetic constructs for conferring herbicide tolerance in cotton, the amount of boll retention is a measure of efficacy and is a desirable trait.

The qualities of selected phenotype displayed by the test organisms or by the test parts are then compared to provide a ranked order of the individual codons, encoding a specific amino acid, that are tandemly repeated in the synthetic constructs according to their preference of usage by the organism or part to confer the selected phenotype. One of ordinary skill in the art will thereby be able to determine a “codon preference table” for each amino acid in the polypeptide whose expression conveys the selected phenotype to the organism of interest. Comparison of synonymous codons within a codon preference table can then be used to identify codons for tailoring a synthetic polynucleotide to modulate the quality of a'selected phenotype.

3. Selection of Synonymous And First Codons

In accordance with the present invention, a comparison of phenotypic preferences can be determined for synonymous codons in an organism of interest or in a related organism, or in test parts thereof, which can be used as a basis for constructing a synthetic polynucleotide from which a phenotype-associated polypeptide is producible in the organism of interest or part thereof. The synthetic polynucleotide is constructed so that its expression in the organism or part confers a selected phenotype upon that organism or part but in a different quality than that conferred by a parent polynucleotide that encodes the same polypeptide. The method comprises selecting a first codon of the parent polynucleotide for replacement with a synonymous codon, wherein the synonymous codon is selected on the basis that it exhibits a different phenotypic preference than the first codon in a comparison of phenotypic preferences in the organism of interest or in a related organism, or in a part thereof, as determined in Section 2. The first codon is then replaced with the synonymous codon to construct the synthetic polynucleotide.

Thus, in accordance with the present invention, a parent polynucleotide can be modified with synonymous codons such that quality of the selected phenotype conferred by the polynucleotide so modified (synthetic polynucleotide) is higher than from the parent polynucleotide. Generally, the difference between the respective phenotypic qualities conferred by a synthetic polynucleotide and by a parent polynucleotide depends on the number of first codons that are replaced by synonymous codons, and on the difference in phenotypic preference between the first codons and the synonymous codons in the organism of interest or part thereof. Put another way, the fewer such replacements, and/or the smaller the difference in phenotypic preference between the synonymous and first codons, the smaller the difference will be in the phenotypic quality between the synthetic and parent polynucleotides. Conversely, the more such replacements, and/or the greater the difference in phenotypic preference between the synonymous and first codons, the greater the difference will be in the phenotypic quality between the synthetic and parent polynucleotides.

In one embodiment, the present invention contemplates a method of constructing a synthetic polynucleotide that encodes a phenotype-associated polypeptide and that confers a higher quality of a selected phenotype displayed by an organism of interest or part thereof than the quality conferred by a parent polynucleotide that codes for the same polypeptide. In this embodiment, a first codon of the parent polynucleotide is selected for replacement with a synonymous codon, wherein the synonymous codon is selected on the basis that it exhibits a higher phenotypic preference than the first codon in a comparison of phenotypic preferences in the organism of interest or in a related organism, or in a part thereof.

In accordance with the present invention, a higher quality of a selected phenotype can be achieved by selecting a synonymous codon that has a higher phenotypic preference than the first codon. Generally, a higher phenotypic preference will correlate with a higher quality of the selected phenotype. Thus, in a non-limiting example of such a correlation, a synonymous codon is deemed to have at least about a 5% higher phenotypic preference than a first codon when the quality of phenotype displayed by an organism or part thereof to which a synthetic construct comprising a tandem repeat of the synonymous codon has been provided is at least about 5% higher than the quality of phenotype displayed by an organism or part thereof to which a synthetic construct comprising a tandem repeat of the first codon has been provided. When selecting the synonymous codon, it is preferred that it has a phenotypic preference in the organism of interest that is at least about 105%, suitably at least about 110%, preferably at least about 120%, more preferably at least about 130%, even more preferably at least about 140%, even more preferably at least about 150%, even more preferably at least about 160%, even more preferably at least about 170%, even more preferably at least about 180%, even more preferably at least about 190%, even more preferably at least about 200%, even more preferably at least about 250%, even more preferably at least about 300%, even more preferably at least about 350%, even more preferably at least about 400%, even more preferably at least about 450%, even more preferably at least about 500%, even more preferably at least about 550%, even more preferably at least about 600%, even more preferably at least about 650%, and still even more preferably at least about 700% of the phenotypic preference of the first codon. In the case of two or more synonymous codons having similar phenotypic preferences, it will be appreciated that any one of these codons can be used to replace the first codon. Generally, if a parent polynucleotide has a choice of low and intermediate phenotypic preference codons, it is preferable in the first instance to replace some, or more preferably all, of the low phenotypic preference codons with synonymous codons having intermediate, or preferably high, phenotypic preferences. Typically, replacement of low with intermediate or high phenotypic preference codons results in a substantial increase in the quality of the phenotype conferred by the synthetic polynucleotide so constructed. However, it is also preferable to replace some, or preferably all, of the intermediate phenotypic preference codons with high translationally efficient codons for conferring an optimal quality in the selected phenotype.

In another embodiment, the present invention contemplates a method of constructing a synthetic polynucleotide that encodes a phenotype-associated polypeptide and that confers a lower quality of a selected phenotype displayed by an organism of interest or part thereof than the quality conferred by a parent polynucleotide that codes for the same polypeptide. In this embodiment, a first codon of the parent polynucleotide is selected for replacement with a synonymous codon, wherein the synonymous codon is selected on the basis that it exhibits a lower phenotypic preference than the first codon in a comparison of phenotypic preferences in the organism of interest or in a related organism or in a part thereof.

In accordance with the present invention, a lower quality of a selected phenotype can be achieved by selecting a synonymous codon that has a lower phenotypic preference than the first codon. A lower phenotypic preference will typically correlate with a lower quality of the selected phenotype. Accordingly, in a non-limiting example of such a correlation, a synonymous codon is deemed to have at least about a 5% lower phenotypic preference than a first codon when the quality of phenotype displayed by an organism or part thereof to which a synthetic construct comprising a tandem repeat of the synonymous codon has been provided is at least about 5% lower than the quality of phenotype displayed by an organism or part thereof to which a synthetic construct comprising a tandem repeat of the first codon has been provided. When selecting the synonymous codon for this embodiment, it is preferred that it has a phenotypic preference in the organism of interest that is at least about 95%, suitably at least about 90%, preferably at least about 85%, more preferably at least about 80%, even more preferably at least about 75%, even more preferably at least about 70%, even more preferably at least about 65%, even more preferably at least about 60%, even more preferably at least about 55%, even more preferably at least about 50%, even more preferably at least about 45%, even more preferably at least about 40%, even more preferably at least about 35%, even more preferably at least about 30%, even more preferably at least about 25%, even more preferably at least about 20%, even more preferably at least about 15%, even more preferably at least about 10%, and still even more preferably at least about 5% of the phenotypic preference of the first codon.

It is preferable but not necessary to replace all the codons of the parent polynucleotide with synonymous codons having higher or lower phenotypic preference in the organism of interest or part thereof than the first codons. For example, a higher or lower phenotypic quality can be accomplished even with partial replacement. Typically, the replacement step affects 5%, 10%, 15%, 20%, 25%, 30%, more preferably 35%, 40%, 50%, 60%, 70% or more of the first codons of the parent polynucleotide. In a preferred embodiment requiring a higher phenotypic quality, the number of, and difference in phenotypic preference between, the first codons and the synonymous codons are selected such that the phenotype-associated polypeptide is produced from the synthetic polynucleotide to confer a phenotype upon a chosen organism or organism part in a quality that is at least about 110%, suitably at least about 150%, preferably at least about 200%, more preferably at least about 250%, even more preferably at least about 300%, even more preferably at least about 350%, even more preferably at least about 400%, even more preferably at least about 450%, even more preferably at least about 500%, and still even more preferably at least about 1000%, of the quality of phenotype conferred by the parent polynucleotide in the organism or part. Conversely, in a preferred embodiment requiring a lower phenotypic quality, the number of, and difference in phenotypic preference between, the first codons and the synonymous codons are selected such that the phenotype-associated polypeptide is produced from the synthetic polynucleotide to confer a phenotype upon a chosen organism or part thereof in a quality that is no more than about 90%, suitably no more than about 80%, preferably no more than about 70%, more preferably no more than about 60%, even more preferably no more than about 50%, even more preferably no more than about 40%, even more preferably no more than about 30%, even more preferably no more than about 20%, even more preferably no more than about 10%, and still even more preferably no more than about 5%, of the quality of phenotype conferred by the parent polynucleotide in the organism or part.

4. Contstruction of Synthetic Polynucleotides

Replacement of one codon for another can be achieved using standard methods known in the art. For example codon modification of a parent polynucleotide can be effected using several known mutagenesis techniques including, for example, oligonucleotide-directed mutagenesis, mutagenesis with degenerate oligonucleotides, and region-specific mutagenesis. Exemplary in vitro mutagenesis techniques are described for example in U.S. Pat. Nos. 4,184,917, 4,321,365 and 4,351,901 or in the relevant sections of Ausubel, et al. (CURRENT PROTOCOLS IN MOLECULAR BIOLOGY, John Wiley & Sons, Inc. 1997) and of Sambrook, et al., (MOLECULAR CLONING. A LABORATORY MANUAL, Cold Spring Harbor Press, 1989). Instead of in vihro mutagenesis, the synthetic polynucleotide can be synthesised de novo using readily available machinery as described, for example, in U.S. Pat. No. 4,293,652. However, it should be noted that the present invention is not dependent on, and not directed to, any one particular technique for constructing the synthetic polynucleotide.

The parent polynucleotide is suitably a natural gene. However, it is possible that the parent polynucleotide that is not naturally-occurring but has been engineered using recombinant techniques. Parent polynucleotides can be obtained from any suitable source, such as from eukaryotic or prokaryotic organisms, including but not limited to mammals or other animals, and pathogenic organisms such as yeasts, bacteria, protozoa and viruses.

The invention also contemplates synthetic polynucleotides encoding one or more desired portions of the phenotype-associated polypeptide. In this regard, it is preferable that the synthetic polynucleotide encodes at least one functional domain of the phenotype-associated polypeptide, which is preferably at least about 10, more preferably at least about 20, even more preferably at least about 50, even more preferably at least about 100, even more preferably at least about 150, and still more preferably at least about 500 contiguous amino acid residues of that polypeptide.

The invention further contemplates a synthetic construct (or expression vector), comprising a synthetic polynucleotide of the invention, which is operably linked to a regulatory polynucleotide. The regulatory polynucleotide suitably comprises transcriptional and/or translational control sequences, which will be compatible for expression in the organism of interest or in cells of that organism. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the organism of interest or may be derived from an alternative source, where the region is functional in the chosen organism. The choice of promoter will differ depending on the intended host. For example, promoters which could be used for expression in plants include plant promoters such as: constitutive plant promoters examples of which include CaMV35S plant promoter, CaMV19S plant promoter, FMV34S plant promoter, sugarcane bacilliform badnavirus plant promoter, CsVMV plant promoter, Arabidopsis ACT2/ACT8 actin plant promoter, Arabidopsis ubiquitin UBQ1 plant promoter, barley leaf thionin BTH6 plant promoter, and rice actin plant promoter; tissue specific plant promoters examples of which include bean phaseolin storage protein plant promoter, DLEC plant promoter, PHS.beta. plant promoter, zein storage protein plant promoter, conglutin gamma plant promoter from soybean, AT2S1 gene plant promoter, ACT11 actin plant promoter from Arabidopsis, napa plant promoter from Brassica napus and potato patatin gene plant promoter; and inducible plant promoters examples of which include a light-inducible plant promoter derived from the pea rbcS gene, a plant promoter from the alfalfa rbcS gene, DRE, MYC and MYB plant promoters which are active in drought; INT, INPS, prxEa, Ha hsp17.7G4 and RD21 plant promoters active in high salinity and osmotic stress, and hsr203J and str246C plant promoters active in pathogenic stress. Alternatively, promoters which could be used for expression in mammals include the metallothionein promoter, which can be induced in response to heavy metals such as cadmium, the β-actin promoter as well as viral promoters such as the SV40 large T antigen promoter, human cytomegalovirus (CMV) immediate early (IE) promoter, rous sarcoma virus LTR promoter, adenovims promoter, or a HPV promoter, particularly the HPV upstream regulatory region (URR) may also be used. All these promoters are well described and readily available in the art.

The synthetic construct of the present invention may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterised by effecting the addition of polyadenylic acid tracts to the 3′ end of the MnRNA precursor. Polyadenylation signals are commonly recognised by the presence of homology to the canonical form 5′ AATAAA-3′ although variations are not uncommon. The 3′ non-translated regulatory DNA sequence preferably includes from about 50 to 1,000 nucleotide base pairs and may contain transcriptional and translational termination sequences in addition to a polyadenylation signal and any other regulatory signals capable of effecting mRNA processing or gene expression.

In a preferred embodiment, the synthetic construct further contains a selectable marker gene to permit selection of an organism or a precursor thereof that contains the synthetic construct. Selection genes are well known in the art and will be compatible for expression in organism or its precursor.

It will be understood, however, that expression of protein-encoding polynucleotides in heterologous systems is now well known, and the present invention is not directed to or dependent on any particular vector, transcriptional control sequence or technique for its production. Rather, synthetic polynucleotides prepared according to the methods set forth herein may be introduced into an organism of interest or into a precursor or progenitor thereof in any suitable manner in conjunction with any suitable synthetic construct or vector, and the synthetic polynucleotides may be expressed with known promoters in any conventional manner.

In some embodiments, the synthetic constructs of the invention are in the form of viral vectors, such as simian virus 40 (SV40) or bovine papilloma virus (13PV), which has the ability to replicate as extra-chromosomal elements (Eukaryotic Viral Vectors, Cold Spring Harbor Laboratory, Gluzman ed., 1982; Sarver et al., 1981, Mol. Cell. Biol. 1:486). Viral vectors include retroviral (lentivirus), adeno-associated virus (see, e.g., Okada, 1996, Gene Ther. 3:957-964; Muzyczka, 1994, J. Clin. Invst. 94:1351; U.S. Pat. Nos. 6,156,303; 6,143,548 5,952,221, describing AAV vectors; see also U.S. Pat. Nos. 6,004,799; 5,833,993), adenovirus (see, e.g., U.S. Pat. Nos. 6,140,087; 6,136,594; 6,133,028; 6,120,764), reovirus, herpesvirus, rotavirus genomes etc., modified for introducing and directing expression of a polynucleotide or transgene in cells. Retroviral vectors can include those based upon murine leukemia virus (see, e.g., U.S. Pat. No. 6,132,731), gibbon ape leukemia virus (see, e.g., U.S. Pat. No. 6,033,905), simian immuno-deficiency virus, human immuno-deficiency virus (see, e.g., U.S. Pat. No. 5,985,641), and combinations thereof.

Vectors also include those that efficiently deliver genes to animal cells in vivo (e.g., stem cells) (see, e.g., U.S. Pat. Nos. 5,821,235 and 5,786,340; Croyle et al., 1998, Gene Ther. 5:645; Croyle et al., 1998, Pharm. Res. 15:1348; Croyle et al., 1998, Hum. Gene Ther. 9:561; Foreman et al., 1998, Hum. Gene Ther. 9:1313 ; Wirtz et al., 1999, Gut 44:800). Adenoviral and adeno-associated viral vectors suitable for in vivo delivery are described, for example, in U.S. Pat. Nos. 5,700,470, 5,731,172 and 5,604,090. Additional vectors suitable for in vivo delivery include herpes simplex virus vectors (see, e.g., U.S. Pat. No. 5,501,979), retroviral vectors (see, e.g., U.S. Pat. Nos. 5,624,820, 5,693,508 and 5,674,703; and WO92/05266 and WO92/14829), bovine papilloma virus (BPV) vectors (see, e.g., U.S. Pat. No. 5,719,054), CMV-based vectors (see, e.g., U.S. Pat. No. 5,561,063) and parvovirus, rotavirus and Norwalk virus vectors. Lentiviral vectors are useful for infecting dividing as well as non-dividing cells (see, e.g., U.S. Pat. No. 6,013,516).

Vectors for insect cell expression commonly use recombinant variations of baculoviruses and other nucleopolyhedrovirus, e.g., Bombyx mori nucleopolyhedrovirus vectors (see, e.g., Choi, 2000, Arch. Virol. 145:171-177). For example, Lepidopteran and Coleopteran cells are used to replicate baculoviruses to promote expression of foreign genes carried by baculoviruses, e.g., Spodoptera frugiperda cells are infected with recombinant Autographa californica nuclear polyhedrosis viruses (AcNPV) carrying a heterologous, e.g., a human, coding sequence (see, e.g., Lee, 2000, J. Virol. 74:11873-11880; Wu, 2000, J. Biotechnol. 80:75-83). See, e.g., U.S. Pat. No. 6,143,565, describing use of the polydnavirus of the parasitic wasp Glyptapanteles indiensis to stably integrate nucleic acid into the genome of Lepidopteran and Coleopteran insect cell lines. See also, U.S. Pat. Nos. 6,130,074; 5,858,353; 5,004,687.

Expression vectors capable of expressing proteins in plants are well known in the art, and include, e.g., vectors from Agrobacterium spp., potato virus X (see, e.g., Angell, 1997, EMBO J. 16:3675-3684), tobacco mosaic virus (see, e.g., Casper, 1996, Gene 173:69-73), tomato bushy stunt virus (see, e.g., Hillman, 1989, Virology 169:42-50), tobacco etch virus (see, e.g., Dolja, 1997, Virology 234:243-252), bean golden mosaic virus (see, e.g., Morinaga, 1993, Microbiol Immunol. 37:471476), cauliflower mosaic virus (see, e.g., Cecchini, 1997, Mol. Plant Microbe Interact. 10: 1094-1101), maize Ac/Ds transposable element (see, e.g., Rubin, 1997, Mol. Cell. Biol. 17:6294-6302; Kunze, 1996, Curr. Top. Microbiol. Immunol. 204:161-194), and the maize suppressor-mutator (Spm) transposable element (see, e.g., Schlappi, 1996, Plant Mol. Biol. 32:717-725); and derivatives thereof.

The invention further contemplates organisms containing therein the synthetic construct of the invention, or alternatively, parts, precursors, cells or tissues produced by the methods of the invention.

In order that the invention may be readily understood and put into practical effect, particular preferred embodiments will now be described by way of the following non-limiting examples.

### EXAMPLES

### Example 1

### Codon-Modification of an Antigen-Encoding Polynucleotide to Enhance the Antibody Response of a Mammal to that Antigen

### Reagents

For each set of synonymous codons that code for a single amino acid, a set of 2-6 gene constructs is constructed, each encoding green fluorescent protein with a leader sequence comprising six copies of an amino acid. Each member of the set is identical except that, across the set, the leader sequence comprises 6 copies of each of the possible synonymous codon triplets (XX) that code for the amino acid.

The general format of these gene constructs would be:

Eukaryotic Promoter—ATG—(XXX)6—GFP gene—polyadenylation signal

The nucleic acid and deduced amino acids sequences of the GFP gene are set forth in SEQ ID NO: 1 and 2, respectively. The nucleic acid and deduced amino acids sequences of the different leader sequences are set forth in SEQ ID NO: 3 to 120.

A superset of 20 such sets of gene constructs is thus constructed, encoding all possible naturally occurring amino acids for which there are redundant coding triplets, comprising in total 59 gene constructs.

### Method

Groups of 8-10 6 wk old Balb/c female mice are immunised transcutaneously using a biolistic delivery system (e.g., the Biorad gene gun or the Powderject delivery system) with one of the 59 gene constructs, on one to three occasions at three weekly intervals. Plasmid (1-2 μg) are coated onto the gold beads (Biorad) or sugar (Powderject) material according to the manufacturers instructions. Beads are delivered to the shaven abdominal skin of the mouse. The Powderject system is optimised for delivery to the basal epidermis. The Biorad system has the capacity to alter delivery depth by adjusting the gas pressure used for the delivery. Data are collected for gas pressures optimised for delivery to the superficial, or basal epithelium, or to the dermis. This is calibrated for the particular gene gun and beads by delivering beads to skin and examining microscopic sections to score the percentage of beads at various depths in the dermis and epidermis.

Serum samples are taken by retro-orbital bleeding at weeks 3 and 6 post immunisation and by terminal cardiac puncture at week 9 post immunisation and the titre of antibody to GFP determined by ELISA for each serum. For ELISA, GFP (Sigma) is coated onto plates and serum reactivity determined according to standard protocols (see, e.g., Walsh et al., 2000, J Gen Virol. 709-718).

### Outcome and Interpretation

The mean antibody titre to GPF induced by each set of constructs encoding a given amino acid is ranked to determine the optimal codon for antibody production using a polynucleotide delivered by a biolistic delivery system (conversely the optimal codon to avoid antibody production).

A list of optimal codons is thus be produced. An improved gene construct for a polynucleotide vaccine delivered by the tested delivery system to skin and designed to induce maximal antibody titre is one in which codons in the gene of interest (e.g., influenza hernagglutinin) which are not optimal are (in large measure) replaced by ones that are optimal for producing maximal antibody titre.

### Example 2

### Codon-Modification of an Antigen-Encoding Polynucleotide to Confer Tolerance to that Antigen in a Mammal

### Reagents

For each set of synonymous codons that code for a single amino acid, a set of 2-6 gene constructs is constructed, each encoding glutamic acid decarboxylase (GAD) with a leader sequence comprising six copies of an amino acid. Each member of the set is identical except that, across the set, the leader sequence comprises 6 copies of each of the possible synonymous codon triplets (x) that code for the amino acid.

The general format of such gene constructs would be:

Eukaryotic Promoter—ATG—(XXX)6—GAD gene—polyadenylation signal

The nucleic acid and deduced amino acids sequences of the GAD gene are set forth in SEQ ID NO: 121 and 122, respectively.

A superset of 20 such sets of gene constructs is thus constructed, encoding all possible naturally occurring amino acids for which there are redundant coding triplets, comprising in total 59 gene constructs.

### Methods

Groups of 4 week old NOD (non obese diabetic) female mice are immunised transcutaneously using a biolistic delivery system (e.g., the Biorad gene gun or the Powderject delivery system) with one of the 59 gene constructs, on one to three occasions at three weekly intervals. Plasmid (1-2 μg) are coated onto the gold beads (Biorad) or sugar (Powderject) material according to the manufacturers instructions. Beads are delivered to the shaven abdominal skin of the mouse. The Powderject system is optimised for delivery to the basal epidermis. The Biorad system has the capacity to alter delivery depth by adjusting the gas pressure used for the delivery. Data are collected for gas pressures optimised for delivery to the superficial, or basal epithelium, or to the dermis. This is calibrated for the particular gene gun and beads by delivering beads to skin and examining microscopic sections to score the percentage of beads at various depths in the dermis and epidermis.

The mice are followed to the onset of diabetes as assessed by a blood sugar level over 10 mM/L on weekly blood samples obtained by retro-orbital bleeding. Onset of diabetes is confirmed by testing for glycosuria, and pancreatic histology determined on the diabetic mice, and on mice deemed free of diabetes at 26 weeks.

Outcome and Interpretation

The mean time to onset of diabetes for mice immunised with each set of constructs encoding a given amino acid would be ranked, to determine the optimal codon for expressing the gene of interest in the sites responsible for prevention of a pathogenic immune response, using a polynucleotide delivered by a biolistic delivery system.

A list of optimal codons for ensuring protein expression from a polynucleotide vaccine at the optimal sites to induce host protective immunity (tolerance) is thus produced. An improved gene construct for a polynucleotide vaccine delivered by the tested delivery system to skin and designed to induce maximal host protective immune tolerance is one in which codons in the gene of interest (e.g., insulin with a view to controlling human diabetes) which are not optimal are (in large measure) replaced by ones that are optimal.

### Example 3

### Codon-Modification for Optimising the Resistance of Transgenic Plants to Herbicides while Maintaining seed Protein Production

### Reagents

For each set of synonymous codons that code for a single amino acid, a set of 2-6 gene constructs is constructed, each encoding phosphinothricin acetyltransferase (encoded by the bar gene) with a leader sequence comprising six copies of an amino acid. Each member of the set is identical except that, across the set, the leader sequence comprises 6 copies of each of the possible synonymous codon triplets (IX) that code for the amino acid.

The general format of such gene constructs would be:

Plant Promoter—ATG—(XM)6—BAR gene—plant polyadenylation signal

The nucleic acid and deduced amino acids sequences of the BAR gene are set forth in SEQ ID NO: 123 and 124, respectively.

A superset of 20 such sets of gene constructs is thus constructed, encoding all possible naturally occurring amino acids, comprising in total 59 gene constructs.

### Method

The method of Weeks et al. (1993, Plant Physiol 102(4):1077-1084) for the production of multiple independent lines of fertile transgenic wheat (Triticuin aestivum). Briefly, calli derived from immature embryos, 0.5 to 1 mm long, are bombarded with microprojectiles coated with DNA containing as marker genes the bar gene, encoding phosphinothricin-resistance, and the gene encoding β-glucuronidase (GUS), each under control of a maize ubiquitin promoter. The bombardment is performed 5 d after embryo excision, just after initiation of callus proliferation. The ability of plantlets to root in the presence of 1 or 3 mg/L of bialaphos is a reliable selection criteria used to identify transformed plants. Stable transformation is confirmed by marker gene expression assays and the presence of the bar sequences in high molecular weight chromosomal DNA of the resultant plants. On average, 168 d elapses between embryo excision for bombardment and anthesis of the TO plants. The transmission of both the resistance phenotype and bar DNA to the T1 generation verified that germline transformation had occurred.

Herbicide resistance is measured using the method of Singh and Wright (2002, Lett. Appl. Microbiol. 35(1):12-16) and protein concentration in seed is measured by the method of Turner et al. (1990, Plant Mol. Biol. 14(5):793-803).

### Outcome and Interpretation

For each amino acid leader sequence, plants are ranked according to their resistance to the Basta Herbicide, and to their seed protein content. The optimal codon from each set is the one which gives plants adequate herbicide resistance and the maximum seed protein yield.

A list of optimal codons is thus be produced, and an improved gene construct for a gene conveying herbicide resistance to a transgenic grain plant is one in which codons in the gene of interest (say those in the broad host range plasmid pJP4, which carries genes for the degradation of 2,4-dichlorophenoxyacetic acid (2,4-D), 2-methyl-4-chlorophenoxyacetic acid, and 3-chlorobenzoic acid (see e.g., Kleinsteuber, et al, 2001, Extremophiles. 5(6):375-384) which are not optimal are (in large measure ) replaced by ones that are optimal.

### Example 4

### Codon-Modification for Optimising the Selectivity of a Growth Inhibitory Gene for Controlling a Skin Tumour without Damage to Surrounding Normal Skin

### Reagents

For each set of synonymous codons that code for a single amino acid, a set of 2-6 gene constructs is constructed, each encoding an E2F1 dominant negative inhibitor with a leader sequence comprising six copies of an amino acid. Each member of the set is identical except that, across the set, the leader sequence comprises 6 copies of each of the possible synonymous codon triplets (XXX) that code for the amino acid.

The general format of such gene constructs would be:

Eukaryotic Promoter—ATG—(XXX)6—E2F1dn gene—IRES—GFP gene—polyadenylation signal

The nucleic acid and deduced amino acids sequences of the E2F1 dominant negative inhibitor gene are set forth in SEQ ID NO: 125 and 126, respectively.

A superset of 20 such sets of gene constructs is thus constructed, encoding all possible naturally occurring amino acids for which there are redundant coding triplets, comprising in total 59 gene constructs.

### Methods

Tumours are induced on the skin of FVB mice by painting with chemical carcinogens using a standard DMBA/TPA promotion/induction regime. Groups of tumour bearing female mice are treated topically using a biolistic delivery system (e.g., the Biorad gene gun or the Powderject delivery system) with one of the 59 gene constructs, on one to three occasions at three weekly intervals. Plasmid (1-2 μg) are coated onto the gold beads (Biorad) or sugar (Powderject) material according to the manufacturers instructions. Beads are delivered to the shaven abdominal skin of the mouse. The Powderject system is optimised for delivery to the basal epidermis. The Biorad system has the capacity to alter delivery depth by adjusting the gas pressure used for the delivery. Data are collected for gas pressures optimised for delivery to the superficial, or basal epithelium, or to the dermis. This is calibrated for the particular gene gun and beads by delivering beads to skin and examining microscopic sections to score the percentage of beads at various depths in the dermis and epidermis.

Twenty-four hours after treatment, the skin from the tumour and surrounding normal skin is excised and disaggregated into single cells. Cells are sorted for expression of the GFP by high speed flow cytometry, and the clonogenic potential of the GFP positive cells assessed for each construct, and for normal and tumour tissue. Clonogenic potential is established by plating cells on collagen coated plates in minimal KGM medium, and by counting colonies established per 10000 input cells. The ratio of colonies obtained from normal tissue to colonies obtained from tumour tissue is measure of the efficiency of targeting of that particular construct to the tumour tissue—higher colony forming efficiency for the normal skin cells in comparison to the tumour cells indicates a more preferred codon for correctly delivering gene therapy to the tumour.

### Outcome and Interpretation

The mean colony forming efficiency ratio (normal/tumour cells) are ranked to determine the optimal codon for inhibiting tumour growth while preserving normal skin, using a polynucleotide delivered by a biolistic delivery system. A list of optimal codons is thus produced. An improved gene construct for treating skin cancers delivered by the tested delivery system to skin and designed to induce maximal selectivity for the tumour over normal tissue is one in which codons in the gene of interest for skin cancer treatment (for example p53) most resemble the optimal list.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-38. (canceled)

39. A method for constructing a synthetic polynucleotide from which a polypeptide is producible to confer a selected phenotype upon an organism of interest or part thereof in a different quality than that conferred by a parent polynucleotide that encodes the same polypeptide, the method comprising: (a) selecting a first codon of the parent polynucleotide for replacement with a synonymous codon, wherein the synonymous codon is selected on the basis that it exhibits a different phenotypic preference than the first codon in a comparison of phenotypic preferences in test organisms or parts thereof, wherein the test organisms are selected from the group consisting of organisms of the same species as the organism of interest and organisms that are related to the organism of interest; and (b) replacing the first codon with the synonymous codon to construct the synthetic polynucleotide, wherein the selected phenotype is other than a phenotype conferred upon a cell by a polynucleotide that is expressed in the cell and that encodes the polypeptide.

40. A method according to claim 39, wherein the phenotypic preferences of codons in the test organisms or parts are compared by: (i) separately introducing into the test organisms or parts individual synthetic constructs, each of which comprises a regulatory polynucleotide operably linked to a tandem repeat of a codon fused in frame with a reporter polynucleotide that encodes a reporter protein, which produces, or which is predicted to produce, a corresponding phenotype selected from the group consisting of the selected phenotype and a phenotype of the same class as the selected phenotype; and (ii) comparing the quality of the phenotypes displayed by the test organisms or parts to determine the relative phenotypic preferences of the codons.

41. A method according to claim 39, wherein the reporter protein produces the selected phenotype.

42. A method according to claim 39, wherein the reporter protein does not produce the selected phenotype but produces the same class of phenotype as the selected phenotype.

43. A method according to claim 41 or claim 42, wherein the reporter protein is selected from antigens derived from pathogenic organisms, cancer antigens, self antigens, transplantation antigens, growth factors, hormones and toxins.

44. A method according to claim 39, wherein the phenotype is selected from immunity, antigen tolerance, angiogenesis, anti-angiogenesis, amelioration of clinical symptoms, reduced or increased cell death, reduced or increased cell differentiation, reduced or increased cell proliferation, tumour or cancer regression, growth and repair of tissue or organ, decreased fibrosis, inhibition or reversal of cell senescence, increased or reduced cell migration, differential expression of protein between different cells or tissues of an organism or part thereof, trauma recovery, recovery from burns, antibiotic resistance or sensitivity, herbicide tolerance or sensitivity, starch biosynthesis or modification, fatty acid biosynthesis, disease resistance or tolerance, pest resistance or tolerance including insect resistance or tolerance, viral resistance or tolerance, fungal resistance or tolerance, a metabolic trait including sucrose metabolism, frost resistance or tolerance, stress tolerance, and improved food content or increased yields.

45. A method according to claim 39, wherein the phenotype is an immune response.

46. A method according to claim 45, wherein the immune response is a humoral immune response.

47. A method according to claim 45, wherein the immune response is a cell mediated immune response.

48. A method according to claim 45, wherein the immune response is an innate immunity mediated response.

49. A method according to claim 39, wherein the synthetic constructs are introduced into the test organisms using the same or similar mode of introduction.

50. A method according to claim 39, wherein the synthetic constructs are introduced into the test organisms at the same or corresponding site.

51. A method according to claim 39, wherein the organism of interest is a mammal and the synthetic constructs arc introduced by oral, intravenous, intramuscular, intranasal, buccal, subcutaneous, transdermal, buccal or sublingual route.

52. A method according to claim 39, wherein the synthetic constructs are introduced into one or more of cell or tissue types of the organism of interest.

53. A method according to claim 52, wherein the synthetic constructs are introduced into cells selected from muscle cells, skin cells, brain cells, lung cells, kidney cells, pancreas cells, cells of a reproductive organ, heart cells, vascular cells, liver cells, eye cells, flower cells, meristematic cells, root cells and leaf cells.

54. A method according to claim 39, wherein the tandem repeat of each of the synthetic constructs comprises at least three copies of the corresponding codon.

55. A method according to claim 39, wherein the synonymous codon is selected such that it has a higher phenotypic preference than the first codon.

56. A method according to claim 17, wherein the synonymous codon is selected when the quality of the phenotype conferred by the synthetic construct comprising a tandem repeat of the synonymous codon is at least about 5% higher than the quality of the phenotype conferred by the synthetic construct comprising a tandem repeat of the first codon.

57. A method according to claim 39, wherein the synonymous codon is selected such that it has a lower phenotypic preference that the first codon.

58. A method according to claim 57, wherein the synonymous codon is selected when the quality of the phenotype conferred by the synthetic construct comprising a tandem repeat of the synonymous codon is at least about 5% lower than the quality of the phenotype conferred by the synthetic construct comprising a tandem repeat of the first codon.

59. A method according to claim 39, wherein the organism of interest is unicellular.

60. A method according to claim 39, wherein the organism of interest is a multicellular organism.

61. A method according to claim 60, wherein the multicellular organism is an animal.

62. A method according to claim 60, wherein the multicellular organism is a mammal.

63. A method according to claim 60, wherein the multicellular organism is a plant.

64. A method according to claim 39, wherein the synthetic constructs are introduced into progenitors of the test organisms or parts and the progenitors arc grown or cultured for a time and under conditions sufficient to produce the test organisms or parts, whereby the synthetic constructs are contained in one or more cell types of those organisms or parts.

65. A method according to claim 64, wherein the progenitor cells are selected from stem cells, pluripotential cells, meristermatic cells and embryonic callus.

66. A method for determining the phenotypic preference of a first codon in an organism of interest or part thereof, the method comprising: (a) introducing a synthetic construct into a test organism or part thereof, wherein the test organism is selected from the group consisting of an organism of the same species as the organism of interest and an organism that is related to the organism of interest, the synthetic construct comprising a regulatory polynucleotide operably linked to a tandem repeat of the first codon fused in frame with a reporter polynucleotide that encodes a reporter protein, which produces, or which is predicted to produce, a selected phenotype or a phenotype of the same class as the selected phenotype; and (b) determining the quality of the corresponding phenotype displayed by the organism or part, wherein the selected phenotype or the phenotype of the same class as the selected phenotype is other than a phenotype conferred upon a cell by a polynucleotide that is expressed in the cell and that encodes the polypeptide.

67. A method according to claim 66, further comprising: comparing (i) the quality of the corresponding phenotype displayed by a test organism or part thereof to which a synthetic construct comprising a tandem repeat of the first codon was provided; and (ii) the quality of the corresponding phenotype displayed by a test organism or part thereof to which a synthetic construct comprising a tandem repeat of a second codon was provided, wherein the second codon encodes the same amino acid as the first codon, to thereby determine the phenotypic preference of the first codon relative to the phenotypic preference of the second codon in the test organism or part.

68. A method according to claim 66, further comprising: (1) introducing the synthetic construct into a progenitor of a test organism or part thereof; and (2) producing the test organism or part from the progenitor, wherein the test organism or part contains the synthetic construct.

69. A method according to claim 66, further comprising: (1) introducing the synthetic construct into a progenitor of a test organism or part thereof; and (2) growing the test organism or part from the progenitor, wherein the test organism or part comprises a cell containing the synthetic construct.

70. A method according to claim 66, further comprising: introducing the synthetic construct into a selected cell of the test organism or part.

71. A synthetic polynucleotide constructed according to claim 39 or claim 66.

72. An organism of interest or part thereof containing a synthetic polynucleotide constructed according to claim 39 or claim 66.

73. An organism of interest or part thereof containing a synthetic construct that comprises a regulatory polynucleotide operably linked to a tandem repeat of a first codon fused in frame with a reporter polynucleotide that encodes a reporter protein, which produces, or which is predicted to produce, a selected phenotype or a phenotype of the same class as the selected phenotype in the organism or part, wherein the selected phenotype or the phenotype of the same class as the selected phenotype is other than a phenotype conferred upon a cell by a polynucleotide that is expressed in the cell and that encodes the polypeptide.

74. A method of modulating the quality of a selected phenotype that is displayed by an organism of interest or part thereof and that results from the expression of a parent polynucleotide that encodes the polypeptide, the method comprising: introducing into the organism or part a synthetic polynucleotide that is distinguished from the parent polynucleotide by the replacement of a first codon in the parent polynucleotide with a synonymous codon that exhibits a different phenotypic preference than the first codon in the organism or part, wherein the selected phenotype is other than a phenotype conferred upon a cell by a polynucleotide that is expressed in the cell and that encodes the polypeptide.

75. A method of enhancing the quality of a selected phenotype that is displayed by an organism of interest or part thereof and that results from the expression of a parent polynuclcotide that encodes the polypeptide, the method comprising: introducing into the organism or part a synthetic polynucleotide that is distinguished from the parent polynucleotide by the replacement of a first codon in the parent polynucleotide with a synonymous codon that exhibits a higher phenotypic preference than the first codon in the organism or part, wherein the selected phenotype is other than a phenotype conferred upon a cell by a polynucleotide that is expressed in the cell and that encodes the polypeptide.

76. A method of reducing the quality of a selected phenotype that is displayed by an organism of interest or part thereof and that results from the expression of a parent polynucleotide that encodes the polypeptide, the method comprising: introducing into the organism or part a synthetic polynucleotide that is distinguished from the parent polynucleotide by the replacement of a first codon in the parent polynucleotide with a synonymous codon that exhibits a lower phenotypic preference than the first codon in the organism or part, wherein the selected phenotype is other than a phenotype conferred upon a cell by a polynucleotide that is expressed in the cell and that encodes the polypeptide.


SEQUENCE LISTING (126 sequences)

SEQ ID NO: 1 - DNA, 714 bp, Artificial Sequence
  Humanised GFP
  CDS (1)..(711)
agcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccttctcttatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagtga

SEQ ID NO: 2 - protein, 237 aa, Artificial Sequence
  Humanised GFP
SKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - DNA, 18 bp, Artificial Sequence
  Ala(GCA)x6
  CDS (1)..(18)
gcagcagcagcagcagca

SEQ ID NO: 4 - protein, 6 aa, Artificial Sequence
  Ala(GCA)x6
AAAAAA

SEQ ID NO: 5 - DNA, 18 bp, Artificial Sequence
  Ala(GCG)x6
  CDS (1)..(18)
gcggcggcggcggcggcg

SEQ ID NO: 6 - protein, 6 aa, Artificial Sequence
  Ala(GCG)x6
AAAAAA

SEQ ID NO: 7 - DNA, 18 bp, Artificial Sequence
  Ala(GCT)x6
  CDS (1)..(18)
gctgctgctgctgctgct

SEQ ID NO: 8 - protein, 6 aa, Artificial Sequence
  Ala(GCT)x6
AAAAAA

SEQ ID NO: 9 - DNA, 18 bp, Artificial Sequence
  Ala(GCC)x6
  CDS (1)..(18)
gccgccgccgccgccgcc

SEQ ID NO: 10 - protein, 6 aa, Artificial Sequence
  Ala(GCC)x6
AAAAAA

SEQ ID NO: 11 - DNA, 18 bp, Artificial Sequence
  Arg(AGA)x6
  CDS (1)..(18)
agaagaagaagaagaaga

SEQ ID NO: 12 - protein, 6 aa, Artificial Sequence
  Arg(AGA)x6
RRRRRR

SEQ ID NO: 13 - DNA, 18 bp, Artificial Sequence
  Arg(CGA)x6
  CDS (1)..(18)
cgacgacgacgacgacga

SEQ ID NO: 14 - protein, 6 aa, Artificial Sequence
  Arg(CGA)x6
RRRRRR

SEQ ID NO: 15 - DNA, 18 bp, Artificial Sequence
  Arg(CGG)x6
  CDS (1)..(18)
cggcggcggcggcggcgg

SEQ ID NO: 16 - protein, 6 aa, Artificial Sequence
  Arg(CGG)x6
RRRRRR

SEQ ID NO: 17 - DNA, 18 bp, Artificial Sequence
  Arg(CGT)x6
  CDS (1)..(18)
cgtcgtcgtcgtcgtcgt

SEQ ID NO: 18 - protein, 6 aa, Artificial Sequence
  Arg(CGT)x6
RRRRRR

SEQ ID NO: 19 - DNA, 18 bp, Artificial Sequence
  Arg(AGG)x6
  CDS (1)..(18)
aggaggaggaggaggagg

SEQ ID NO: 20 - protein, 6 aa, Artificial Sequence
  Arg(AGG)x6
RRRRRR

SEQ ID NO: 21 - DNA, 18 bp, Artificial Sequence
  Arg(CGC)x6
  CDS (1)..(18)
cgccgccgccgccgccgc

SEQ ID NO: 22 - protein, 6 aa, Artificial Sequence
  Arg(CGC)x6
RRRRRR

SEQ ID NO: 23 - DNA, 18 bp, Artificial Sequence
  Asn(AAC)x6
  CDS (1)..(18)
aacaacaacaacaacaac

SEQ ID NO: 24 - protein, 6 aa, Artificial Sequence
  Asn(AAC)x6
NNNNNN

SEQ ID NO: 25 - DNA, 18 bp, Artificial Sequence
  Asn(AAT)x6
  CDS (1)..(18)
aataataataataataat

SEQ ID NO: 26 - protein, 6 aa, Artificial Sequence
  Asn(AAT)x6
NNNNNN

SEQ ID NO: 27 - DNA, 18 bp, Artificial Sequence
  Asp(GAT)x6
  CDS (1)..(18)
gatgatgatgatgatgat

SEQ ID NO: 28 - protein, 6 aa, Artificial Sequence
  Asp(GAT)x6
DDDDDD

SEQ ID NO: 29 - DNA, 18 bp, Artificial Sequence
  Asp(GAC)x6
  CDS (1)..(18)
gacgacgacgacgacgac

SEQ ID NO: 30 - protein, 6 aa, Artificial Sequence
  Asp(GAC)x6
DDDDDD

SEQ ID NO: 31 - DNA, 18 bp, Artificial Sequence
  Cys(TGC)x6
  CDS (1)..(18)
tgctgctgctgctgctgc

SEQ ID NO: 32 - protein, 6 aa, Artificial Sequence
  Cys(TGC)x6
CCCCCC

SEQ ID NO: 33 - DNA, 18 bp, Artificial Sequence
  Cys(TGT)x6
  CDS (1)..(18)
tgttgttgttgttgttgt

SEQ ID NO: 34 - protein, 6 aa, Artificial Sequence
  Cys(TGT)x6
CCCCCC

SEQ ID NO: 35 - DNA, 18 bp, Artificial Sequence
  Gln(CAA)x6
  CDS (1)..(18)
caacaacaacaacaacaa

SEQ ID NO: 36 - protein, 6 aa, Artificial Sequence
  Gln(CAA)x6
QQQQQQ

SEQ ID NO: 37 - DNA, 18 bp, Artificial Sequence
  Gln(CAG)x6
  CDS (1)..(18)
cagcagcagcagcagcag

SEQ ID NO: 38 - protein, 6 aa, Artificial Sequence
  Gln(CAG)x6
QQQQQQ

SEQ ID NO: 39 - DNA, 18 bp, Artificial Sequence
  Glu(GAA)x6
  CDS (1)..(18)
gaagaagaagaagaagaa

SEQ ID NO: 40 - protein, 6 aa, Artificial Sequence
  Glu(GAA)x6
EEEEEE

SEQ ID NO: 41 - DNA, 18 bp, Artificial Sequence
  Glu(GAG)x6
  CDS (1)..(18)
gaggaggaggaggaggag

SEQ ID NO: 42 - protein, 6 aa, Artificial Sequence
  Glu(GAG)x6
EEEEEE

SEQ ID NO: 43 - DNA, 18 bp, Artificial Sequence
  Gly(GGA)x6
  CDS (1)..(18)
ggaggaggaggaggagga

SEQ ID NO: 44 - protein, 6 aa, Artificial Sequence
  Gly(GGA)x6
GGGGGG

SEQ ID NO: 45 - DNA, 18 bp, Artificial Sequence
  Gly(GGG)x6
  CDS (1)..(18)
gggggggggggggggggg

SEQ ID NO: 46 - protein, 6 aa, Artificial Sequence
  Gly(GGG)x6
GGGGGG

SEQ ID NO: 47 - DNA, 18 bp, Artificial Sequence
  Gly(GGC)x6
  CDS (1)..(18)
ggcggcggcggcggcggc

SEQ ID NO: 48 - protein, 6 aa, Artificial Sequence
  Gly(GGC)x6
GGGGGG

SEQ ID NO: 49 - DNA, 18 bp, Artificial Sequence
  Gly(GGT)x6
  CDS (1)..(18)
ggtggtggtggtggtggt

SEQ ID NO: 50 - protein, 6 aa, Artificial Sequence
  Gly(GGT)x6
GGGGGG

SEQ ID NO: 51 - DNA, 18 bp, Artificial Sequence
  His(CAC)x6
  CDS (1)..(18)
caccaccaccaccaccac

SEQ ID NO: 52 - protein, 6 aa, Artificial Sequence
  His(CAC)x6
HHHHHH

SEQ ID NO: 53 - DNA, 18 bp, Artificial Sequence
  His(CAT)x6
  CDS (1)..(18)
catcatcatcatcatcat

SEQ ID NO: 54 - protein, 6 aa, Artificial Sequence
  His(CAT)x6
HHHHHH

SEQ ID NO: 55 - DNA, 18 bp, Artificial Sequence
  Ile(ATC)x6
  CDS (1)..(18)
atcatcatcatcatcatc

SEQ ID NO: 56 - protein, 6 aa, Artificial Sequence
  Ile(ATC)x6
IIIIII

SEQ ID NO: 57 - DNA, 18 bp, Artificial Sequence
  Ile(ATT)x6
  CDS (1)..(18)
attattattattattatt

SEQ ID NO: 58 - protein, 6 aa, Artificial Sequence
  Ile(ATT)x6
IIIIII

SEQ ID NO: 59 - DNA, 18 bp, Artificial Sequence
  Ile(ATA)x6
  CDS (1)..(18)
ataataataataataata

SEQ ID NO: 60 - protein, 6 aa, Artificial Sequence
  Ile(ATA)x6
IIIIII

SEQ ID NO: 61 - DNA, 18 bp, Artificial Sequence
  Leu(CTC)x6
  CDS (1)..(18)
ctcctcctcctcctcctc

SEQ ID NO: 62 - protein, 6 aa, Artificial Sequence
  Leu(CTC)x6
LLLLLL

SEQ ID NO: 63 - DNA, 18 bp, Artificial Sequence
  Leu(TTG)x6
  CDS (1)..(18)
ttgttgttgttgttgttg

SEQ ID NO: 64 - protein, 6 aa, Artificial Sequence
  Leu(TTG)x6
LLLLLL

SEQ ID NO: 65 - DNA, 18 bp, Artificial Sequence
  Leu(CTA)x6
  CDS (1)..(18)
ctactactactactacta

SEQ ID NO: 66 - protein, 6 aa, Artificial Sequence
  Leu(CTA)x6
LLLLLL

SEQ ID NO: 67 - DNA, 18 bp, Artificial Sequence
  Leu(CTG)x6
  CDS (1)..(18)
ctgctgctgctgctgctg

SEQ ID NO: 68 - protein, 6 aa, Artificial Sequence
  Leu(CTG)x6
LLLLLL

SEQ ID NO: 69 - DNA, 18 bp, Artificial Sequence
  Leu(TTA)x6
  CDS (1)..(18)
ttattattattattatta

SEQ ID NO: 70 - protein, 6 aa, Artificial Sequence
  Leu(TTA)x6
LLLLLL

SEQ ID NO: 71 - DNA, 18 bp, Artificial Sequence
  Leu(CTT)x6
  CDS (1)..(18)
cttcttcttcttcttctt

SEQ ID NO: 72 - protein, 6 aa, Artificial Sequence
  Leu(CTT)x6
LLLLLL

SEQ ID NO: 73 - DNA, 18 bp, Artificial Sequence
  Lys(AAG)x6
  CDS (1)..(18)
aagaagaagaagaagaag

SEQ ID NO: 74 - protein, 6 aa, Artificial Sequence
  Lys(AAG)x6
KKKKKK

SEQ ID NO: 75 - DNA, 18 bp, Artificial Sequence
  Lys(AAA)x6
  CDS (1)..(18)
aaaaaaaaaaaaaaaaaa

SEQ ID NO: 76 - protein, 6 aa, Artificial Sequence
  Lys(AAA)x6
KKKKKK

SEQ ID NO: 77 - DNA, 18 bp, Artificial sequence
  Phe(TTT)x6
  CDS (1)..(18)
tttttttttttttttttt

SEQ ID NO: 78 - protein, 6 aa, Artificial sequence
  Phe(TTT)x6
FFFFFF

SEQ ID NO: 79 - DNA, 18 bp, Artificial Sequence
  Phe(TTC)x6
  CDS (1)..(18)
ttcttcttcttcttcttc

SEQ ID NO: 80 - protein, 6 aa, Artificial Sequence
  Phe(TTC)x6
FFFFFF

SEQ ID NO: 81 - DNA, 18 bp, Artificial Sequence
  Pro(CCC)x6
  CDS (1)..(18)
cccccccccccccccccc

SEQ ID NO: 82 - protein, 6 aa, Artificial Sequence
  Pro(CCC)x6
PPPPPP

SEQ ID NO: 83 - DNA, 18 bp, Artificial Sequence
  Pro(CCT)x6
  CDS (1)..(18)
cctcctcctcctcctcct

SEQ ID NO: 84 - protein, 6 aa, Artificial Sequence
  Pro(CCT)x6
PPPPPP

SEQ ID NO: 85 - DNA, 18 bp, Artificial Sequence
  Pro(CCG)x6
  CDS (1)..(18)
ccgccgccgccgccgccg

SEQ ID NO: 86 - protein, 6 aa, Artificial Sequence
  Pro(CCG)x6
PPPPPP

SEQ ID NO: 87 - DNA, 18 bp, Artificial sequence
  Pro(CCA)x6
  CDS (1)..(18)
ccaccaccaccaccacca

SEQ ID NO: 88 - protein, 6 aa, Artificial sequence
  Pro(CCA)x6
PPPPPP

SEQ ID NO: 89 - DNA, 18 bp, Artificial Sequence
  Ser(AGC)x6
  CDS (1)..(18)
agcagcagcagcagcagc

SEQ ID NO: 90 - protein, 6 aa, Artificial Sequence
  Ser(AGC)x6
SSSSSS

SEQ ID NO: 91 - DNA, 18 bp, Artificial Sequence
  Ser(TCT)x6
  CDS (1)..(18)
tcttcttcttcttcttct

SEQ ID NO: 92 - protein, 6 aa, Artificial Sequence
  Ser(TCT)x6
SSSSSS

SEQ ID NO: 93 - DNA, 18 bp, Artificial Sequence
  Ser(AGT)x6
  CDS (1)..(18)
agtagtagtagtagtagt

SEQ ID NO: 94 - protein, 6 aa, Artificial Sequence
  Ser(AGT)x6
SSSSSS

SEQ ID NO: 95 - DNA, 18 bp, Artificial Sequence
  Ser(TCG)x6
  CDS (1)..(18)
tcgtcgtcgtcgtcgtcg

SEQ ID NO: 96 - protein, 6 aa, Artificial Sequence
  Ser(TCG)x6
SSSSSS

SEQ ID NO: 97 - DNA, 18 bp, Artificial Sequence
  Ser(TCA)x6
  CDS (1)..(18)
tcatcatcatcatcatca

SEQ ID NO: 98 - protein, 6 aa, Artificial Sequence
  Ser(TCA)x6
SSSSSS

SEQ ID NO: 99 - DNA, 18 bp, Artificial Sequence
  Ser(TCC)x6
  CDS (1)..(18)
tcctcctcctcctcctcc

SEQ ID NO: 100 - protein, 6 aa, Artificial Sequence
  Ser(TCC)x6
SSSSSS

SEQ ID NO: 101 - DNA, 18 bp, Artificial Sequence
  Thr(ACA)x6
  CDS (1)..(18)
acaacaacaacaacaaca

SEQ ID NO: 102 - protein, 6 aa, Artificial Sequence
  Thr(ACA)x6
TTTTTT

SEQ ID NO: 103 - DNA, 18 bp, Artificial Sequence
  Thr(ACG)x6
  CDS (1)..(18)
acgacgacgacgacgacg

SEQ ID NO: 104 - protein, 6 aa, Artificial Sequence
  Thr(ACG)x6
TTTTTT

SEQ ID NO: 105 - DNA, 18 bp, Artificial Sequence
  Thr(ACT)x6
  CDS (1)..(18)
actactactactactact

SEQ ID NO: 106 - protein, 6 aa, Artificial Sequence
  Thr(ACT)x6
TTTTTT

SEQ ID NO: 107 - DNA, 18 bp, Artificial Sequence
  Thr(ACC)x6
  CDS (1)..(18)
accaccaccaccaccacc

SEQ ID NO: 108 - protein, 6 aa, Artificial Sequence
  Thr(ACC)x6
TTTTTT

SEQ ID NO: 109 - DNA, 18 bp, Artificial Sequence
  Tyr(TAC)x6
  CDS (1)..(18)
tactactactactactac

SEQ ID NO: 110 - protein, 6 aa, Artificial Sequence
  Tyr(TAC)x6
YYYYYY

SEQ ID NO: 111 - DNA, 18 bp, Artificial Sequence
  Tyr(TAT)x6
  CDS (1)..(18)
tattattattattattat

SEQ ID NO: 112 - protein, 6 aa, Artificial Sequence
  Tyr(TAT)x6
YYYYYY

SEQ ID NO: 113 - DNA, 18 bp, Artificial Sequence
  Val(GTG)x6
  CDS (1)..(18)
gtggtggtggtggtggtg

SEQ ID NO: 114 - protein, 6 aa, Artificial Sequence
  Val(GTG)x6
VVVVVV

SEQ ID NO: 115 - DNA, 18 bp, Artificial Sequence
  Val(GTT)x6
  CDS (1)..(18)
gttgttgttgttgttgtt

SEQ ID NO: 116 - protein, 6 aa, Artificial Sequence
  Val(GTT)x6
VVVVVV

SEQ ID NO: 117 - DNA, 18 bp, Artificial Sequence
  Val(GTC)x6
  CDS (1)..(18)
gtcgtcgtcgtcgtcgtc

SEQ ID NO: 118 - protein, 6 aa, Artificial Sequence
  Val(GTC)x6
VVVVVV

SEQ ID NO: 119 - DNA, 18 bp, Artificial Sequence
  Val(GTA)x6
  CDS (1)..(18)
gtagtagtagtagtagta

SEQ ID NO: 120 - protein, 6 aa, Artificial Sequence
  Val(GTA)x6
VVVVVV

SEQ ID NO: 121 - DNA, 2583 bp, Mouse
  CDS (1)..(2166)
gaacttcgggacgagcaaactccgggccacaggaagaacccatcgaaccaaagcagcttagaatctgactccaattacccctccatttccacttccgaaatcggagacactgaggatgcccttcagcaggtggaggagattggcatagagaaggcagccatggacatgaccgtcttcctgaagctgcagaagagagtgcgcgaacttgagcaggagaggaagaagctgcaggcgcagctagaaaagggacagcaggacagcaagaaagggcaggtagaacaacagaacaatggcttagatgtggaccaggacgcagatatagcctacaatagtctgaagagacaggagcttgagtcagagaacaagaagctgaagaatgacctgaatgagctgaggaacggtgtcgctgaccaagccatgcaggataactccacccacagctccccagacagctacagcctcctactgaaccagctcaagctggccaatgaggagctcgaggtccgcaaagaggaggcgctgatcctcaggacccagatcatgaatgccgaccagcgccgcctgtctggcaagaacatggagccgaacatcaatgccagaacaagttggcccaacagtgagaagcacgtggaccaggaagacgccattgaggcctatcacggggtctgccagacaaacaggttgctggaggcccagctgcaggcccagagcctggagcatgaggaggaggtggaacatctcaaggcccaggtggaagccctgaaagaggagatggacaaacagcagcagaccttctgccagaccctgctgctctccccagaggcccaggtagaatttggtgtccagcaggagatatcccggctgaccaatgagaacctggattttaaggaattggtggaaaagctggagaagaatgagaggaagctgaagaagcagctgaagatttacatgaagaaggtccaggacttagaagctgcccaggcgttggcacagagtgacaggaggcaccatgaactcacaagacaggtcacagtccaacgaaaagagaaggacttccaaggcatgctggagtaccacaaagaggtcgaagccctcctcatccggaacctggtgacagacctgaagcctcagatgctgctgggcaccgtgccctgtctgcctgcatacatactctatatgtgcatcaggcacgcggattacaccaacgatgacctcaaggtgcactcgttgctgagctccaccatcaacggcattaagaaagtcctcaagaagcacaatgacgactttgagatgacgtcattctggttatccaacacctgccgcttccttcactgtctgaagcaatacagtggtgatgagggtttcatgacacagaacatcgcgaagcagaatgagcactgtctcaagaactttgacctcactgaataccgccaggtactaagcgacctttccattcagatctatcagcagctcattaaaatgcccgagggcttgctacagcctatgatagtttctgccatgttggaaaatgagagtatccaggggctgtctggtgtgagaccaactggttaccggaagcgctcctccagcatggtggatggagagaattctttccatacagtcctgtgtgaccagggcctggaccccgagattatcctgcaggtgttcaaacagctcttctacatgatcaatgctgtgactcttaacaacctactcctgcggaaagacgcctgctcctggagcacaggcatgcaactcaggtacaacataagtcaactggaagagtggcttcggggcaaaaaccttcaccagagtggagcagttcagaccatggagcccctgatccaggcagcccagctcctccagctgaagaagaaaacccacgaggatgctgaggccatctgctctctgtgcacctccctcagcacccagcagattgtcaaaattttaaacctctacactcccttgaatgaatttgaggaacgggtcacagtgtccttcatcagaacaatccaggctcagctacaagagaggaatgaccctcagcagctcctgctggactccaagcacgtgttcccagttctgtttccatataacccatctgctctgaccatggactcgatccacatcccggcctgtctcaacctggagtttctcaatgaagtctgaggatgcgtgtttccgaggcgagcgagaaggaagcatgtgctgtcagccgagagaatgctaggtgtgttaaatattccagcgtagatcaaaccatgttagagactggcgggacgacagaactaaacagcggggtgcacagttgtcgccaatgctgctcagaaaacacccggaagtggatttgttaaagctgtgctttcaggttaaaccaagacacgtcagaacgaacagccactctgcagctccagtcgccatataaaaatgccagttctacagagtggaagtgcctagctttgatctttgtatatatcttgagaatgttcaaactgagataatattaaaaacacatgacgtaaattgcctttgtgggtctttcaagaaatgatgggactaataaccataagattgacaggaatt

SEQ ID NO: 122 - protein, 722 aa, Mouse
ELRDEQTPGHRKNPSNQSSLESDSNYPSISTSEIGDTEDALQQVEEIGIEKAAMDMTVFLKLQKRVRELEQERKKLQAQLEKGQQDSKKGQVEQQNNGLDVDQDADIAYNSLKRQELESENKKLKNDLNELRNGVADQAMQDNSTHSSPDSYSLLLNQLKLANEELEVRKEEALILRTQIMNADQRRLSGKNMEPNINARTSWPNSEKHVDQEDAIEAYHGVCQTNRLLEAQLQAQSLEHEEEVEHLKAQVEALKEEMDKQQQTFCQTLLLSPEAQVEFGVQQEISRLTNENLDFKELVEKLEKNERKLKKQLKIYMKKVQDLEAAQALAQSDRRHHELTRQVTVQRKEKDFQGMLEYHKEVEALLIRNLVTDLKPQMLLGTVPCLPAYILYMCIRHADYTNDDLKVHSLLSSTINGIKKVLKKHNDDFEMTSFWLSNTCRFLHCLKQYSGDEGFMTQNIAKQNEHCLKNFDLTEYRQVLSDLSIQIYQQLIKMPEGLLQPMIVSAMLENESIQGLSGVRPTGYRKRSSSMVDGENSFHTVLCDQGLDPEIILQVFKQLFYMINAVTLNNLLLRKDACSWSTGMQLRYNISQLEEWLRGKNLHQSGAVQTMEPLIQAAQLLQLKKKTHEDAEAICSLCTSLSTQQIVKILNLYTPLNEFEERVTVSFIRTIQAQLQERNDPQQLLLDSKHVFPVLFPYNPSALTMDSIHIPACLNLEFLNEV

SEQ ID NO: 123 - DNA, 549 bp, Artificial Sequence
  BAR gene
  CDS (1)..(549)
agcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatctga

SEQ ID NO: 124 - protein, 182 aa, Artificial Sequence
  BAR gene
SPERRPADIRRATEADMPAVCTIVNHYIETSTVNFRTEPQEPQEWTDDLVRLRERYPWLVAEVDGEVAGIAYAGPWKARNAYDWTAESTVYVSPRHQRTGLGSTLYTHLLKSLEAQGFKSVVAVIGLPNDPSVRMHEALGYAPRGMLRAAGFKHGNWHDVGFWQLDFSLPVPPRPVLPVTEI

SEQ ID NO: 125 - DNA, 366 bp, Human
  CDS (1)..(363)
atgggaaaaggtgtgaaatccccgggggagaagtcacgctatgagacctcactgaatctgaccaccaagcgcttcctggagctgctgagccactcggctgacggtgtcgtcgacctgaactgggctgccgaggtgctgaaggtgcagaagcggcgcatctatgacatcaccaacgtccttgagggcatccagctcattgccaagaagtccaagaaccacatccagtggctgggcagccacaccacagtgggcgtcggcggacggcttgaggggttgacccaggacctccgacagctgcaggagagcgagcagcagctggaccacctgatgaatatctgtactacgcagctgcgcctgctctcctga

SEQ ID NO: 126 - protein, 121 aa, Human
MGKGVKSPGEKSRYETSLNLTTKRFLELLSHSADGVVDLNWAAEVLKVQKRRIYDITNVLEGIQLIAKKSKNHIQWLGSHTTVGVGGRLEGLTQDLRQLQESEQQLDHLMNICTTQLRLLS